(19) United States
(12) Patent Application Publication
(10) Pub. No.: US 2024/0398349 A1
(43) Pub. Date: Dec. 5, 2024

(54) CONTACT DETECTION FOR A WEARABLE DEVICE

(71) Applicant: Oura Health Oy, Oulu (FI)

(72) Inventors: Jukka Tapani Mäkinen, Oulu (FI); Mika Petteri Kangas, Oulu (FI); Jaakko Tapio Vartiainen, Oulu (FI); Kirsi Marja Maansaari, Oulu (FI); Olli Petteri Heikkinen, Oulu (FI)

(21) Appl. No.: 18/325,545

(22) Filed: May 30, 2023

(51) Int. Cl. A61B 5/00; A61B 5/024

(52) U.S. Cl. CPC A61B 5/6843; A61B 5/02427; A61B 5/6802; A61B 5/7235; A61B 5/742

(57) ABSTRACT

Methods, systems, and devices for contact detection for a wearable device are described. The system may measure, by the one or more optical components, a photoplethysmogram (PPG) signal for a user of the wearable device and detect a change in the PPG signal or a change in morphology between cardiac pulse waveforms that are based on the PPG signal and reference cardiac pulse waveforms. In some cases, the system may determine, based on the change in the PPG signal or the change in morphology coinciding with a change in a metric measured by the wearable device, that the change in the PPG signal or the change in morphology is based on a change in contact between the wearable device and the skin of the user of the wearable device. The system may output the PPG signal and an indication of the change in contact.

### FIELD OF TECHNOLOGY

The following relates to wearable devices and data processing, including contact detection for a wearable device.

### BACKGROUND

Some wearable devices may be configured to collect data from users so that health and wellness information can be determined for the users. The accuracy of the health and wellness information determined for a user may vary with the reliability of the data collected by a wearable device. Improved techniques for determining the reliability of data collected by wearable devices may be desired.

### BRIEF DESCRIPTION OF THE DRAWINGS

FIG. 1 illustrates an example of a system that supports contact detection for a wearable device in accordance with aspects of the present disclosure.

FIG. 2 illustrates an example of a system that supports contact detection for a wearable device in accordance with aspects of the present disclosure.

FIG. 3 shows an example of wearable device diagrams that supports contact detection for a wearable device in accordance with aspects of the present disclosure.

FIG. 4 shows an example of a timing diagram that supports contact detection for a wearable device in accordance with aspects of the present disclosure.

FIG. 5 shows an example of a timing diagram that supports contact detection for a wearable device in accordance with aspects of the present disclosure.

FIG. 6 shows an example of a process flow that supports contact detection for a wearable device in accordance with aspects of the present disclosure.

FIG. 7 shows a block diagram of an apparatus that supports contact detection for a wearable device in accordance with aspects of the present disclosure.

FIG. 8 shows a block diagram of a wearable device manager that supports contact detection for a wearable device in accordance with aspects of the present disclosure.

FIG. 9 shows a diagram of a system including a device that supports contact detection for a wearable device in accordance with aspects of the present disclosure.

FIG. 10 shows a block diagram of an apparatus that supports contact detection for a wearable device in accordance with aspects of the present disclosure.

FIG. 11 shows a block diagram of a wearable application that supports contact detection for a wearable device in accordance with aspects of the present disclosure.

FIG. 12 shows a diagram of a system including a device that supports contact detection for a wearable device in accordance with aspects of the present disclosure.

FIGS. 13 through 15 show flowcharts illustrating methods that support contact detection for a wearable device in accordance with aspects of the present disclosure.

### DETAILED DESCRIPTION

A wearable device may collect data, such as physiological data, from a user that can be used to determine health and wellness information for the user. For example, the wearable device may collect the data via one or more sensors that couple with (e.g., interface with, contact) the skin of the user. The reliability (e.g., accuracy) of the data collected by the wearable device may be contact-dependent, meaning that the reliability may vary with the contact between the wearable device and the skin of the user. But the contact between the wearable device and the skin of the user may be inconsistent (e.g., due to external forces exerted against the wearable device, changes in finger position, changes in finger girth, and the like).

Use of unreliable data collected while the contact between the wearable device is poor may result in inaccurate health and wellness information for the user. For example, using data while the contact between the wearable device and the user changes may result in inaccurate physiological data readings, which may lead to a distorted picture of the user's overall health, as well as increased power consumption and decreased battery life.

Accordingly, to facilitate improved physiological measurements, aspects of the present disclosure are directed to using photoplethysmogram (PPG) data and a second metric to detect a change in contact between the user and the wearable device. As such, the reliability of data collected by a wearable device may be determined based on the contact between the wearable device and the user. To determine the contact between the wearable device and the user, a device (e.g., the wearable device, a user device, a server) may evaluate one or more characteristics derived from a PPG signal generated by the wearable device. For example, the device may evaluate the level of the PPG signal or the morphology of cardiac pulse waveforms generated from the PPG signal, along with one or more other metrics measured by the wearable device, to determine whether a disturbance in the optical signal is due to poor contact.

In such cases, the wearable device may measure, by one or more optical components, the PPG signal and detect a change in the PPG signal or a change in morphology between cardiac pulse waveforms. The cardiac pulse waveforms may be based on the PPG signal and reference cardiac pulse waveforms. The wearable device may determine that the change in the PPG signal or the change in morphology is based on a change in contact between the wearable device and the skin of a user of the wearable device. In such cases, the wearable device may identify a change in contact between the wearable device and the skin of the user. In some cases, the change in morphology between cardiac pulse waveforms may coincide with a change in a metric measured by the wearable device. The wearable device may output the PPG signal, an indication of the change in contact, or both.

Identifying durations of poor contact may allow the wearable device to detect unreliable data, among other advantages. For example, detecting unreliable data from poor contact may allow the wearable device to output an indication to the user to change the contact between the wearable device and the user, which may lead to more accurate physiological data measurements as the contact between the wearable device and the user is improved, thereby increasing the efficiency and accuracy of the signal.

Aspects of the disclosure are initially described in the context of systems supporting physiological data collection from users via wearable devices. Aspects of the disclosure are further described in the context of wearable ring device diagrams, timing diagrams, and a process flow: Aspects of the disclosure are further illustrated by and described with reference to apparatus diagrams, system diagrams, and flowcharts that relate to contact detection for a wearable device.

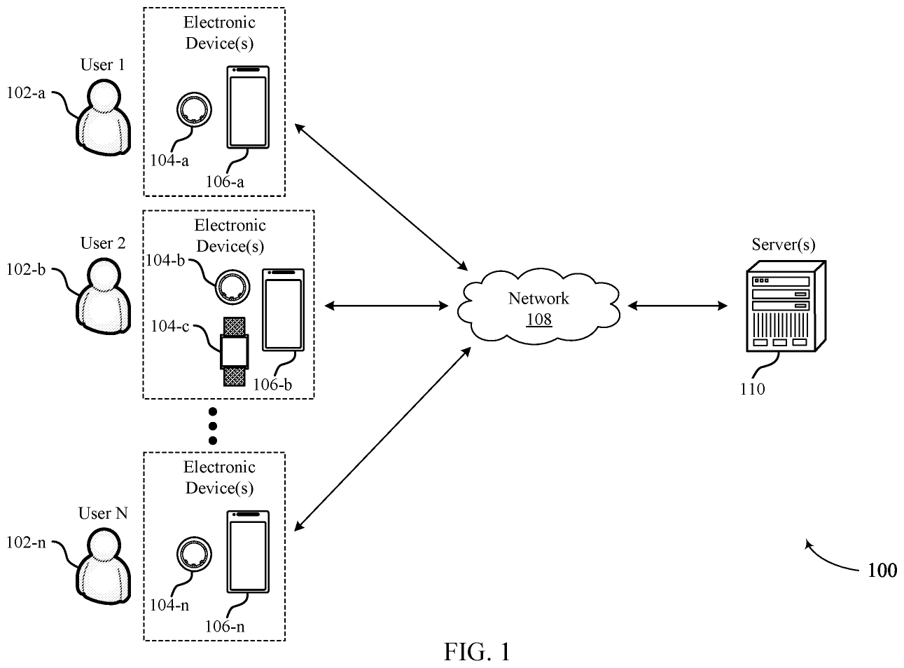

FIG. 1 illustrates an example of a system 100 that supports contact detection for a wearable device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via graphical user interfaces (GUIs)) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state: 2) circadian rhythms: 3) non-endogenous daily rhythms shown to be imposed on top of circadian rhythms, as in work schedules: 4) weekly rhythms, or other artificial time periodicities exogenously imposed (e.g. in a hypothetical culture with 12 day “weeks,” 12 day rhythms could be used): 5) multi-day ovarian rhythms in women and spermatogenesis rhythms in men: 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for detecting contact for a wearable device. As noted, a wearable device 104 may interface with the skin of a user 102 to collect data that is used to determine health and wellness information for the user 102. For example, a wearable ring device may interface with the skin of a user's finger to collect PPG data that is used to determine health metrics for the user such as blood pressure, blood oxygen level, and HRV. But the accuracy of the data generated by the wearable device 104 may vary with the contact between the wearable device 104 and the user, which in turn may vary over time. For example, there may be durations of time in which the contact between the wearable device 104 and the user 102 is insufficient (or excessive) for the collection of accurate data. If periods of poor contact go undetected, inaccurate data collected during the periods of poor contact may be used to generate inaccurate health and wellness information for the user 104.

According to the techniques described herein, a device (e.g., a wearable device 104, a user device 106, the server 110) may use a signal (e.g., a PPG signal) and one or more other metrics measured by a wearable ring device 104 to detect periods of poor contact between the wearable device 104 and a user 102. Upon detecting a period of poor contact, the device may prompt the user 102 to adjust the orientation of wearable device 104 and/or may adjust processing of data collected by the wearable device 104 during the period to account or compensate for the poor contact.

For example, the system 100 may measure the PPG signal for a user of the wearable device and detect a change in the PPG signal or a change in morphology between cardiac pulse waveforms that are based on the PPG signal and reference cardiac pulse waveforms. The system 100 may determine, based on the change in the PPG signal or the change in morphology, that the change in the PPG signal or the change in morphology is based on a change in contact between the wearable device and the skin of a user of the wearable device. The change in the PPG signal or the change in morphology may coincide with a change in the metric (e.g., characteristics of the cardiac pulse waveforms, skin or body temperature, motion, light measurements, etc.) measured by the wearable device. The system 100 may output the PPG signal and an indication of the change in contact.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

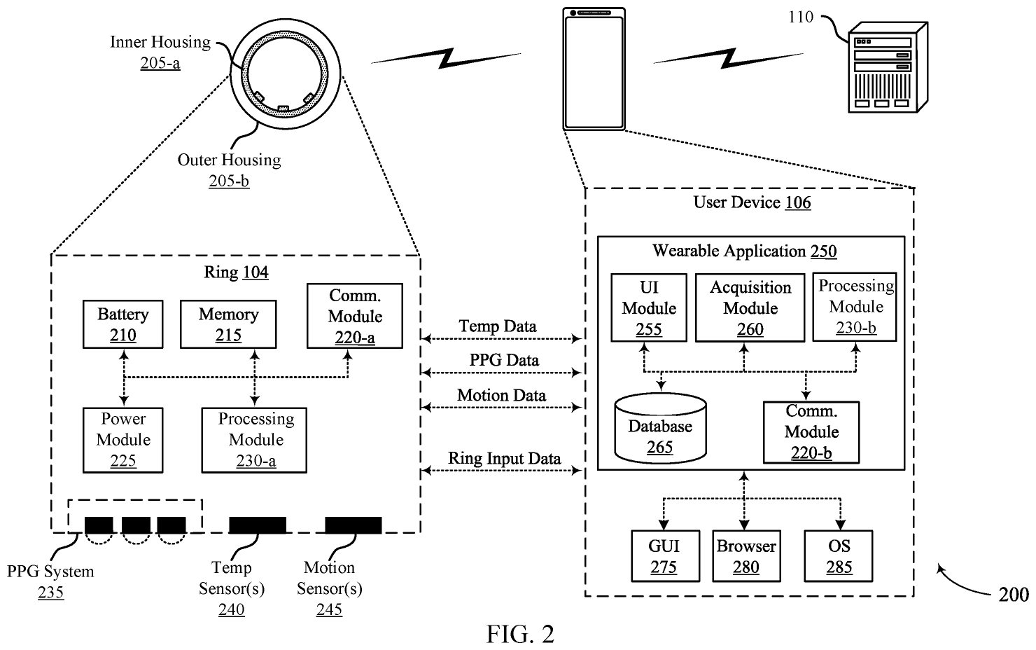

FIG. 2 illustrates an example of a system 200 that supports contact detection for a wearable device in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep day's may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency.” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support a wearable device that detects a change in contact between the wearable device and the skin of the user based on a change in the PPG signal or a change in the morphology between cardiac pulse waveforms. In particular, techniques described herein support a ring 104, such as a wearable device 104 as described with reference to FIG. 1. For example, a ring 104 may include an inner housing 205-a configured to house a sensor module that includes one or more sensors that are configured to acquire physiological data from a user 102. The one or more sensors of the ring 104 may obtain physiological measurements from the user (e.g., temperature sensors, additional LED-PD sensors used for measuring heart rate, oxygen saturation, one or more sensors that a device may use to detect whether a user is asleep, or the like).

In some cases, the one or more sensors of the ring 104 are configured to acquire the physiological data from the user based on arterial blood flow, temperature, etc. In some implementations, the one or more sensors of the ring 104 are configured to acquire the physiological data (e.g., including the PPG data) from the user based on blood flow that is diffused into the microvascular bed of skin with capillaries and arterioles. The one or more sensors of the ring 104 may be an example of photodetectors from the PPG system 235, temperature sensors 240, motion sensors 245, galvanic sensors, and other sensors.

As noted, various physiological metrics (e.g., blood pressure, blood oxygen level, HRV) determined by the ring 104 may be based on a PPG signal measured by the ring 104. The reliability (e.g., accuracy) of the PPG signal may be based on the contact between the optical components (e.g., LEDs, photodetectors, lenses, windows) of the ring 104 and the skin of the user. For example, the PPG signal measured by the ring 104 may not accurately reflect physiological aspects of the user if the optical components of the ring 104 are too tightly pressed against, or are separated from, the skin of the user. According to the techniques described herein, a device may use a PPG signal, potentially with other metrics measured by the ring 104, to detect when contact between the ring 104 and the skin of the user is ill-suited for accurate data-collection.

While much of the present disclosure describes one or more components in the context of a wearable ring device, aspects of the present disclosure may additionally or alternatively be implemented in the context of other wearable devices. For example, in some implementations, the one or more components described herein may be implemented in the context of other wearable devices, such as bracelets, watches, necklaces, piercings, and the like. For example, the wearable device 104 may surround a finger, wrist, ankle, earlobe, or the like of a user.

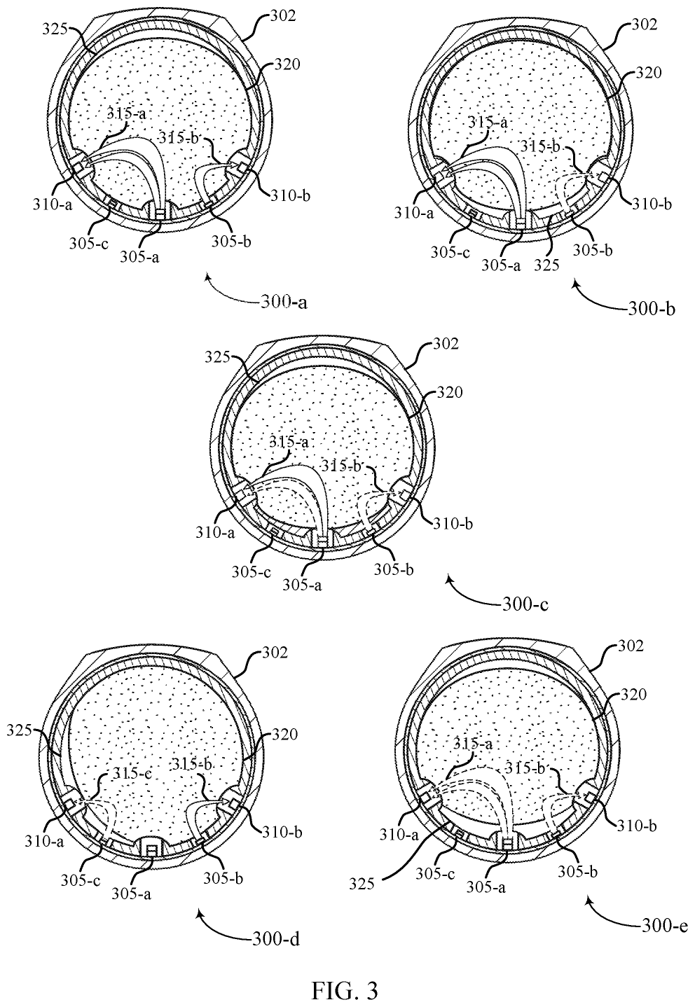

FIG. 3 shows an example of wearable device diagrams 300 that support contact detection for a wearable device in accordance with aspects of the present disclosure. The wearable device diagrams 300 may include wearable devices 302, which may include optical components such as light-emitting components 305 (e.g., LEDs, vertical-cavity surface-emitting lasers (VCELs)) and optical receivers 310 (e.g., photosensors, phototransistors, and photodiodes).

The optical receivers 310 may be configured to measure PPG signals based on light 315 received from the light-emitting components 305. For example, the optical receivers 310-a and 310-b may be configured to measure PPG signals based on red light 315-a and green light 315-b, respectively. The light-emitting component 305-a may emit the red light 315-a, and the light emitting component 305-b may emit the green light 315-b. Other colors (e.g., wavelengths) of light are contemplated and within the scope of the present disclosure.

The light 315 received from the light-emitting component 305 may vary with the contact between the user's finger 320 and the optical components, which in turn may cause the PPG signal to fluctuate. For example, a gap 325 (e.g., an airgap) between the skin of the user and the wearable device 302 may disturb the optical path (e.g., because the light 315 is now coupled to the finger 320 through an additional interface) such that some or all of the light 315 is directed away from the optical receivers 310. Additionally or alternatively, a material (e.g., water, oil, dirt) in the gap 325 may dampen or absorb the light 315, resulting in less light 315 reaching the optical receivers 310. In such cases, the light coupling between the optical components (e.g., the light-emitting components 305) and the user's finger 320 may change as the distribution of light 315 into the skin changes and the reflections at the optical interface changes.

In some cases, skin contact between the user's finger 320 and the wearable device 302 may vary due to an external force pushing on the wearable device 302, a gap 325 between the user's finger 320 and the wearable device 302, liquid or contaminants between the user's finger 320 and the wearable device 302, or a combination thereof. Changes in skin contact or pressure against the user's finger 320 may affect physiological measurements. For example, the changes in skin contact and/or pressure may cause inaccuracy in the physiological measurements and higher battery consumption. In some examples, the wearable device 302 may move around the user's finger 320, causing rotation of the wearable device 302 and losing contact between one or more optical components that may affect the physiological measurements.

In some cases, some or all of the green light 315-b may be directed away from the optical receiver 310-b, as described with reference to wearable device diagrams 300-b, 300-c, and 300-e. For example, the gap 325 (e.g., an airgap) between the skin of the user and the wearable device 302 may disturb the optical path (e.g., because the light 315 is now coupled to the finger 320 through an additional interface) such that some or all of the green light 315-b is directed away from the optical receivers 310-b. In some cases, the gap 325 may not affect the red light 315-a such that the red light 315-a may be directed towards the optical receiver 310-a. Wearable device diagram 300-b may be an example of a wearable device 302 that receives an external pressure from a top surface of the wearable device 302. The gap 325 may form between an inner, bottom surface of the wearable device 302 and the user's finger 320.

In some cases, some or all of the red light 315-a may be directed away from the optical receiver 310-a, as described with reference to wearable device diagrams 300-c and 300-e. For example, the gap 325 between the skin of the user and the wearable device 302 may disturb the optical path such that some or all of the red light 315-a is directed away from the optical receiver 310-a and some or all of the green light 315-b is directed away from the optical receiver 310-b. Wearable device diagram 300-c may be an example of a wearable device 302 that receives an external pressure from a bottom surface of the wearable device 302 such that the gap 325 is formed between an inner, top surface of the wearable device 302 and the user's finger 320.

The wearable device diagram 300-d may include a gap between the inner, bottom left surface of the wearable device 302 and the user's finger 320. In such cases, the gap 325 between the skin of the user and the wearable device 302 may disturb the optical path such that some or all of the green light 315-c is directed away from the optical receiver 310-a and some or all of the green light 315-b is directed towards the optical receiver 310-b. The external forces on the wearable device 302 may push one side of the optical components away from the user's finger 320 to create the gap 325.

If the gap 325 between the wearable device 302 and the skin is sufficiently substantial or filled with a certain material, some or all of the light 315 (e.g., including light 315-a and 315-b) may be directed away from the optical receivers 310, as described with reference to wearable device diagram 300-e. For example, liquid or contaminants (e.g., dirt, and the like) may be trapped between the user's finger 320 and the wearable device 302. The optical signals may be dampened or totally absorbed by the material trapped between the wearable device 302 and the user's finger 320. The difference in refractive indexes and the material layer may determine how the different optical signal paths may be affected. In some cases, the wearable device diagram 300-e may be an example of an overall poor fit of the wearable device 302 such that one or more gaps 325 may be present between the user's finger 320 and the wearable device 302. In some cases, all of the light 315 may be directed away from the optical receivers 310.

In some cases, the skin contact lost between the user's finger 320 and the wearable device 302 may not affect the physiological measurements. For example, as described with reference to wearable device diagram 300-a, the gap 325 between an inner, top surface of the wearable device 302 and the user's finger 320 may not affect the physiological measurements. The optical receivers 310-a and 310-b may be configured to measure PPG signals based on red light 315-a and green light 315-b, respectively, as the light 315 is directed towards the optical receivers 310.

Because the PPG signals generated by the wearable device 302 are based on the light 315 received by the optical receivers 310, variations in the light 315 received by the optical receivers 310 may change the PPG signals. If a device (e.g., the wearable device 302) is unable to detect when changes in PPG signals are attributed to poor contact between the wearable device 302 and the user, the device may indiscriminately use the PPG signals to determine inaccurate physiological metrics from the user. According to the techniques described herein, a device may use changes in a PPG signal, along with one or more other metrics, to determine when changes in the PPG signal are due to poor skin contact. In some cases, identifying an impact on the physiological metrics from losing skin contact between the user's finger 320 and the wearable device 302 may enable the wearable device 302 to change measurement channels to an alternative side of the wearable device 302 where the wearable device 302 contacts the user's finger 320.

The device may look for certain changes in the PPG signal (or a cardiac pulse waveform generated therefrom) as a condition for evaluating the other metric(s) indicative of poor contact. For example, the device may wait until the direct current (DC) level of the PPG signal changes by a threshold amount within a threshold duration of time before evaluating the other metrics. As another example, the device may wait until detecting a morphology difference between cardiac pulse waveforms generated from PPG signal before evaluating the other metrics. Thus, the device may evaluate the other metrics based on detecting a change in the PPG signal (or cardiac pulse waveforms generated from the PPG signal).

Together, the other metric(s) and the change in the PPG signal (or cardiac pulse waveforms generated from the PPG signal) may provide enough information for the device to determine whether a change in the PPG signal (or cardiac pulse waveforms generated from the PPG signal) is attributable to poor skin contact (rather than reflecting an actual physiological change in the user). For example, the device may determine that a change in the PPG signal (or cardiac pulse waveforms generated from the PPG signal) is attributable to poor skin contact if the change in PPG signal (or cardiac pulse waveforms) temporally coincides with (e.g., is concurrent with, at least partially overlaps in time with) a change in the other metric(s).

In some examples, the other metric may be the light reflected into one or more laser components, such as VCSELs. A laser component may be disposed along the inner circumference of the wearable device, similar to the other optical components, and may emit a laser beam into the skin of the user so that a portion of light from the laser is reflected back into the laser component. If the light reflected into the laser component changes coincident with a change in a PPG signal (or a cardiac pulse waveform generated therefrom), the device may determine that the change in the PPG signal (or the cardiac pulse waveform) is based on (e.g., due at least in part to) a change in skin contact.

In some examples, the other metric may be measured by one or more galvanic sensors that are configured to detect skin contact. The galvanic sensor(s) may be galvanic surface(s) that are disposed along the inner circumference of the wearable device 302. If the galvanic sensors detect a threshold change in skin contact coincident with a change in a PPG signal (or a cardiac pulse waveform generated therefrom), the device may determine that the change in the PPG signal (or the cardiac pulse waveform) is based on the change in skin contact. In some examples, the galvanic sensors may be conductive ring-shaped coatings that circumnavigate the apertures of the optical components. Other configurations of the galvanic sensors are contemplated and within the scope of the present disclosure.

The galvanic sensors (e.g., galvanic skin response (GSR) sensors) may utilize at least two electrically conductive electrodes that contact the skin surface. In some cases, the electrodes that are not fixated to (e.g., contacting) the skin may move around which affects the contact area between the electrodes and the skin. In such cases, the GSR signal may include a plurality of minimums and maximums with a greater amplitude and frequency than skin conductance responses (SCRs).

In some cases, the wearable device 302 may measure an amount of ambient light received at the optical receivers 310. In such cases, the amount of ambient light received at the optical receivers 310 may be an example of the other metric. If there is a threshold change in an amount of ambient light measured (e.g., an increase or decrease) coincident with a change in a PPG signal (or a cardiac pulse waveform generated therefrom), the wearable device 302 may determine that the change in the PPG signal (or the cardiac pulse waveform) is based on (e.g., due at least in part to) a change in skin contact.

In some cases, the amount of ambient light received at the optical receivers 310 may increase. For example, the optical receivers 310 may detach from the skin (e.g., due to movement) and an air gap (e.g., gap 325) may form between the wearable device 302 and the optical receivers 310, thereby allowing ambient light to leak into the optical receivers 310 from outside the wearable device 302. The ambient light levels may be measured periodically by the wearable device 302 during specific time intervals when the light-emitting components 305 (e.g. LEDs) are not powered.

In some examples, the other metric may be a change or difference in amplitude between cardiac pulse waveforms that are generated based on the PPG signal. If the change in amplitude satisfies a threshold and is coincident with a change in a PPG signal (or a cardiac pulse waveform generated therefrom), the device may determine that the change in the PPG signal (or the cardiac pulse waveform) is based on (e.g., due at least in part to) a change in skin contact.

In some examples, the other metric may be temperature measured by the wearable device 302. The temperature may be measured by one or more temperature sensors disposed along the inner circumference of the wearable device 302. If there is a threshold change in temperature (e.g., an increase in temperature, a decrease in temperature) coincident with a change in a PPG signal (or a cardiac pulse waveform generated therefrom), the device may determine that the change in the PPG signal (or the cardiac pulse waveform) is based on (e.g., due at least in part to) a change in skin contact.

In some examples, the other metric may be acceleration measured by the wearable device 302. The acceleration may be measured by an accelerometer or similar motion sensor disposed within the wearable device 302. If there is a threshold change in acceleration coincident with a change in a PPG signal (or a cardiac pulse waveform generated therefrom), the device may determine that the change in the PPG signal (or the cardiac pulse waveform) is based on (e.g., due at least in part to) a change in skin contact.

In some examples, the other metric may be orientation measured by the wearable device 302. The orientation may be measured by a gyroscope or similar orientation sensors disposed within the wearable device 302. If there is a threshold change in orientation coincident with a change in a PPG signal (or a cardiac pulse waveform generated therefrom), the device may determine that the change in the PPG signal (or the cardiac pulse waveform) is based on (e.g., due at least in part to) a change in skin contact.

In some examples, the device may compare the data (e.g., PPG signals) measured by different combinations of optical components to detect when there is poor contact between the user and the wearable device and/or determine information about (such as the location of) the poor contact. For example, the device may compare the PPG signal associated with a first optical path (e.g., between the red light 315-a and the optical receiver 310-a) with the PPG signal associated with a second optical path (e.g., between the green light 315-b and the optical receiver 310-b) to determine that the wearable device is pressed too tightly against the left side of the user's finger 320.

Thus, a device may use the relative timing of multiple metrics to determine when changes in physiological data measured by the wearable device 302 are due to changes in skin contact between the wearable device 302 and the user as opposed to being actually representative of underlying physiological changes (e.g., the heart rate changing).

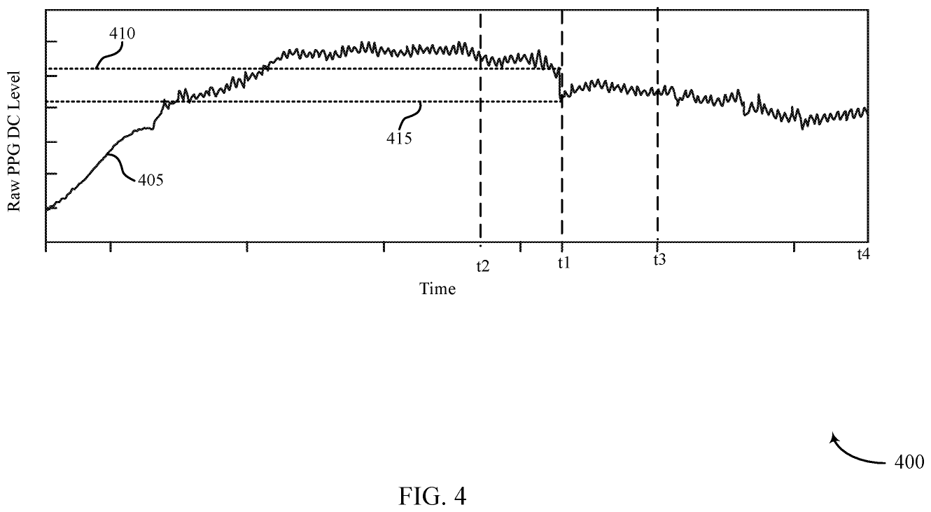

FIG. 4 shows an example of a timing diagram 400 that supports contact detection for a wearable device in accordance with aspects of the present disclosure. The timing diagram 400 may implement, or be implemented by, aspects of the system 100, system 200, wearable device diagram 300, or a combination thereof. The timing diagram 400 may include a PPG signal 405. The PPG signal 405 may be an example of a PPG signal that is measured by a wearable device as described herein. The PPG signal 405 may have a DC level (indicated by the y-axis) that changes over time (indicated by the x-axis).

A device may monitor or analyze the DC level of the PPG signal 405 to detect when changes in physiological data measured by the device are due to changes in skin contact. For example, the device may monitor or analyze the PPG signal 405 to detect anomalous events in which the PPG signal 405 changes a threshold amount within a threshold duration. For example, the device may determine that the PPG signal 405 has an anomalous event at time t1, when the PPG signal decreases a threshold amount from DC level 410 to DC level 415 within a threshold duration of time.

At time t1, the sudden drop in DC level may be caused by the sensor detaching from skin, thereby indicating a change in contact between the wearable device and the user. The DC component of PPG signal 405 may vary with many physiological and external physical processes. The DC levels of the PPG signal 405 may be monitored and discontinuities over a certain threshold may detect, for example, the discontinuity at time t1. Detection of an anomalous event may trigger the device to evaluate one or more other metrics measured by the wearable device to determine whether the anomalous event is due to a change in contact or some other phenomenon. The one or more other metrics measured by the wearable device may be an example of a change in temperature, a change in motion, a change in acceleration, a change in ambient light absorbed, and the like.

For example, the device may determine that the DC level of the PPG signal 405 has changed by a threshold amount within a threshold duration of time in response to detecting the change in the PPG signal 405. In some cases, the device may exclude a portion of the PPG signal 405 from a calculation to determine a physiological metric for the user. In such cases, the PPG signal 405 may be filtered to discard some information contained in the raw PPG signal 405. For example, the device may exclude a portion of the PPG signal 405 at time t1, when the PPG signal decreases a threshold amount from DC level 410 to DC level 415 within a threshold duration of time. In other examples, the device may exclude a portion of the PPG signal 405 from time t2 to time t3 when the PPG signal 405 decreases a threshold amount within a threshold duration of time. In some cases, further signal processing and algorithms may be used for extraction of the feature parameters associated with the PPG signal 405.

In other examples, the device may adjust the signal processing to refrain from excluding the one or more portions of the PPG signal 405 when the PPG signal decreases a threshold amount. For example, the device may apply calibration coefficients in the signal processing algorithms to adjust the calculations without excluding the one or more portions of the PPG signal 405 when the PPG signal decreases a threshold amount. In some cases, the device may switch and use different calibration coefficients in the signal processing algorithms based on an amount (e.g., degree) that the PPG signal 405 decreases.

The PPG signal 405 may include undulations. However, the drop in DC level at time t1 may indicate a loss of skin contact between the wearable device and the user. In some cases, a sudden increase in the DC level of the PPG signal 405 may indicate a change in contact between the wearable device and the user. The sudden change in DC level of the PPG signal 405 may affect the signal quality for SpO2 measurements as the DC level of the PPG signal 405 may be included in SpO2 calculations.

After time t1, the DC level of the PPG signal 405 may continue to decrease. In such cases, the device may determine an affected range of data and determine the portion of the PPG signal 405 that may be excluded from the calculation to determine a physiological metric for the user. In some cases, the device may apply a weighting factor to the portion of the PPG signal 405 based on the portion of the PPG signal 405 coinciding with the change in contact. In such cases, the device may apply the weighting factor to the portion of the PPG signal at time t1, from time t2 to time t3, from time t2 to time t4, from time t1 to t4, or any combination thereof.

The morphology of the PPG signal 405 may be calculated in real-time. In other examples, the PPG signal 405 may be stored and the morphology of the PPG signal 405 may be calculated based on retrieving the PPG signal 405 from storage. In some cases, the discontinuity of the PPG signal 405 at time t1 may affect physiological measurements such as blood pressure, SpO2, heart rate, and the like. In some examples, the discontinuity of the PPG signal 405 at time t1 may affect calculations of cardiovascular age. In some cases, the device may identify a portion of the PPG signal 405 that maintains a same DC level. In other cases, the device may identify a portion of the PPG signal 405 that changes DC levels.

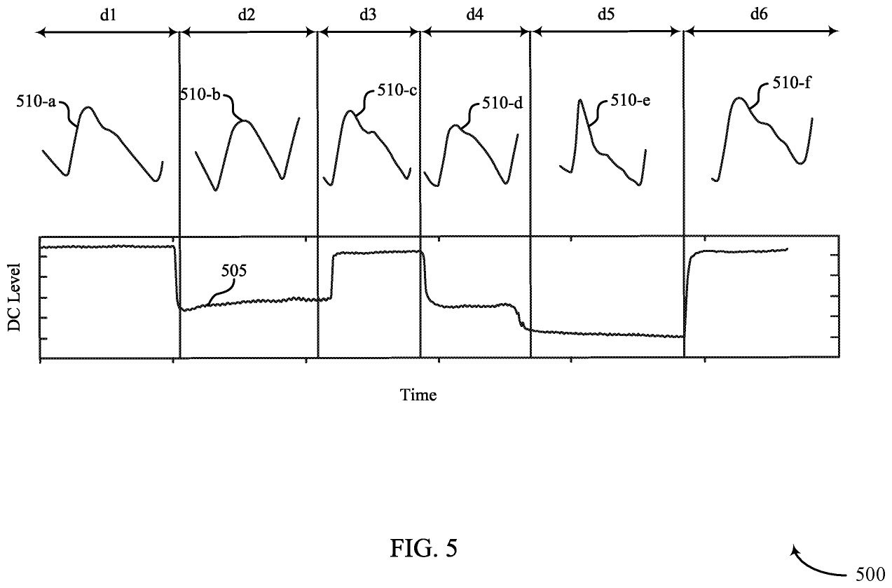

FIG. 5 shows an example of a timing diagram 500 that supports contact detection for a wearable device in accordance with aspects of the present disclosure. The timing diagram 500 may implement, or be implemented by, aspects of the system 100, system 200, wearable device diagram 300, timing diagram 400, or a combination thereof. The timing diagram 500 may include a PPG signal 505 and cardiac pulse waveform 510.

The PPG signal 505 may be an example of a PPG signal that is measured by a wearable device as described herein. The PPG signal 505 may have a DC level (indicated by the y-axis) that changes over time (indicated by the x-axis). The cardiac pulse waveforms 510 may include cardiac pulse waveforms generated from the PPG signal 505 at different times.

For example, the cardiac pulse waveforms 510 may include at least cardiac pulse waveform 510-a, which may be generated from the PPG signal 505 measured during duration d1. The cardiac pulse waveform 510-a and PPG signal 505 measured during duration d1 may be representative of the contact and/or pressure between the wearable device and the user's finger when the finger is straight. The cardiac pulse waveform 510-a may include at least two local maximums and a dicrotic notch.

The cardiac pulse waveform 510-b may be generated from the PPG signal 505 measured during duration d2. The cardiac pulse waveform 510-b and PPG signal 505 measured during duration d2 may be representative of the contact and/or pressure between the wearable device and the user's finger when the finger is bent. In some cases, a pressure between the wearable device and the finger of the user may have an effect on the DC level of the PPG signal 505, the morphology of the cardiac pulse waveform 510, or both. In such cases, a loss of contact between the wearable device and the finger of the user may have an effect on the DC level of the PPG signal 505, the morphology of the cardiac pulse waveform 510, or both.

Bending the finger may increase a pressure between the wearable device and the finger as the contact between the wearable device and the finger changes. For example, the contact between the wearable device and the user may increase. The diameter of the finger may increase as the finger bends and the tissue is pressed inside the wearable device. The blood pressure flowing through the finger may also increase as the finger bends. The tissue, blood flow, and blood pressure may change with the changing compression. The blood flow in the arteries and/or capillaries may be occluded when bending the finger. In such cases, the pressure under the sensors of the wearable device may increase, and the occlusion may affect PPG measurements.

As the pressure and/or contact between the wearable device and the finger of the user increases during duration d2, the PPG signal 505 may decrease as compared to the PPG signal 505 measured during duration d1. The morphology of the cardiac pulse waveform 510-b may change as compared to the cardiac pulse waveform 510-a. In some cases, the system may detect a change in the PPG signal 505 based on the PPG signal and reference cardiac pulse waveforms 510.

The cardiac pulse waveform 510-a may be an example of a reference cardiac pulse waveform. In such cases, the system may detect a change in morphology between cardiac pulse waveforms 510-a and 510-b that are based on the PPG signal 505 and reference cardiac pulse waveforms. In such cases, the system may determine, based on the change in the PPG signal 505, that the change in the PPG signal 505 is based on a change in contact between the wearable device and the skin of a user of the wearable device as the finger bends during duration d2. The system may determine, based on the change in morphology, that the change in morphology of cardiac pulse waveform 510-b is based on a change in contact between the wearable device and the skin of a user of the wearable device as the finger bends during duration d2.

The cardiac pulse waveform 510-c may be generated from the PPG signal 505 measured during duration d3. The cardiac pulse waveform 510-c and PPG signal 505 measured during duration d3 may be representative of the contact and/or pressure between the wearable device and the user when the finger is straight, as described with reference to the PPG signal 505 measured during duration d1 and the cardiac pulse waveform 510-a.

The system may detect a change in the morphology of the cardiac pulse waveform 510-c as compared to the cardiac pulse waveform 510-b. In such cases, the system may determine, based on the change in the PPG signal 505 or the change in morphology, that the change in the PPG signal 505 or the change in morphology of the cardiac pulse waveform 510-c is based on a change in contact between the wearable device and the skin of a user of the wearable device as the finger is changed from a bent position to a straight position. In such cases, the system may determine that the contact between the wearable device and the skin of the user of the wearable device is different during duration d2 and duration d3.

In some cases, the system may detect that the morphology of the cardiac pulse waveform 510-c is the same as the cardiac pulse waveform 510-a. The system may detect that the PPG signal 505 measured during duration d3 is the same as the PPG signal 505 measured during duration d1. In such cases, the system may determine that the contact between the wearable device and the skin of the user of the wearable device is the same during duration d1 and duration d3.

The cardiac pulse waveform 510-d may be generated from the PPG signal 505 measured during duration d4. The cardiac pulse waveform 510-d and PPG signal 505 measured during duration d4 may be representative of the contact and/or pressure between the wearable device and the user when the finger is pushed from below. Applying pressure to the wearable ring device as the finger is pushed from below may increase a pressure between the wearable device and the finger as the contact between the wearable device and the finger changes. The cardiac pulse waveform 510-d and the PPG signal 505 measured during duration d4 may be an example of signals derived from the wearable device diagram 300-c, as described with reference to FIG. 3

The contact between the wearable device and the user may increase as the pressure increases from pushing the finger from below. In some cases, the compression of the tissue inside the wearable device may increase as the blood pressure increases and the blood flow decreases. For example, the blood flow in the arteries may be occluded when pushing the finger from below as well as increasing the pressure under the sensors of the wearable device. In such cases, the occlusion may affect PPG measurements and decrease the DC level of the PPG signal 505 during duration d4.

As the pressure and/or contact between the wearable device and the finger of the user increases during duration d4 as compared to duration d3, the PPG signal 505 may decrease. The morphology of the cardiac pulse waveform 510-d may change as compared to the cardiac pulse waveform 510-c. The system may detect a change in morphology between cardiac pulse waveforms 510-d and 510-c that are based on the PPG signal 505 and cardiac pulse waveform 510-c. The cardiac pulse waveform 510-c may be an example of the reference cardiac pulse waveform as the finger is straight.

In such cases, the system may determine, based on the change in the PPG signal 505 during duration d4, that the change in the PPG signal 505 is based on a change in contact between the wearable device and the skin of a user of the wearable device as the finger is pushed from below 4. The system may determine, based on the change in morphology, that the change in morphology of cardiac pulse waveform 510-d is based on a change in contact between the wearable device and the skin of a user of the wearable device as the finger is pushed from below during duration d4.

The cardiac pulse waveform 510-e may be generated from the PPG signal 505 measured during duration d5. The cardiac pulse waveform 510-e and PPG signal 505 measured during duration d5 may be representative of the contact and/or pressure between the wearable device and the user when the finger is pushed from below with a pressure greater than the pressure applied during duration d4. In such cases, the DC level of the PPG signal 505 may be less than the DC level of the PPG signal 505 measured during duration d4 as the pressure and the contact increases.

The system may detect a change in morphology between cardiac pulse waveforms 510-e and 510-d that are based on the PPG signal 505 and reference cardiac pulse waveform (e.g., cardiac pulse waveform 510-a or 510-c). In such cases, the system may determine, based on the change in the PPG signal 505, that the change in the PPG signal 505 is based on a change in contact between the wearable device and the skin of a user of the wearable device as the finger is pushed from below harder during duration d5 as compared to duration d4. The system may determine, based on the change in morphology, that the change in morphology of cardiac pulse waveform 510-e is based on a change in contact between the wearable device and the skin of a user of the wearable device as the finger is pushed below at a greater pressure during duration d5 than duration d4.

The cardiac pulse waveform 510-f may be generated from the PPG signal 505 measured during duration d6. The cardiac pulse waveform 510-f and PPG signal 505 measured during duration d6 may be representative of the contact and/or pressure between the wearable device and the user when the finger is straight, as described with reference to the PPG signal 505 measured during duration d1 and duration d3 (e.g., and including the cardiac pulse waveforms 510-a and 510-c). In such cases, the cardiac pulse waveform 510-f may be compared to previous cardiac pulse waveforms 510, and the PPG signal 505 during duration d6 may be compared with the previous PPG signals 505 during previous durations, as described previously herein.

In some cases, the morphology of the cardiac pulse waveform 510 may change without a change in the PPG signal 505. For example, the cardiac pulse waveform 510-f may vary as compared to the cardiac pulse waveform 510-a and/or 510-c while the PPG signal 505 stays the same.

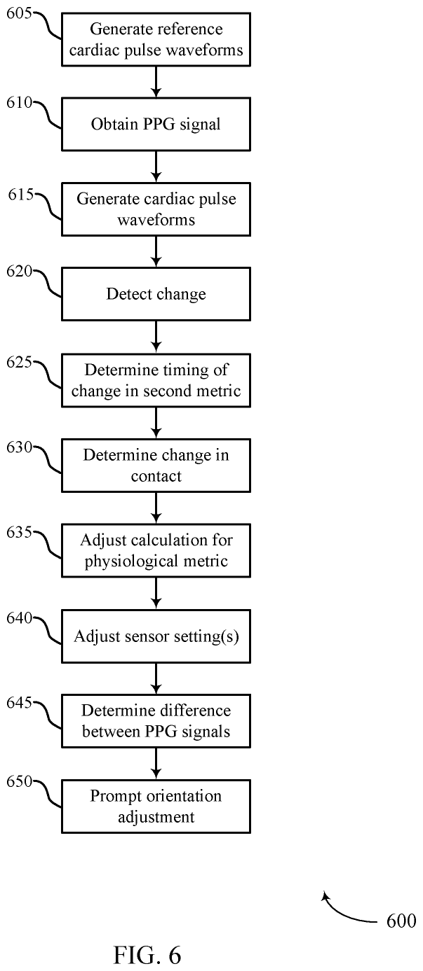

FIG. 6 shows an example of a process flow 600 that supports contact detection for a wearable device in accordance with aspects of the present disclosure. The process flow 600 may be implemented by system 100 and system 200 including at least a server 110, a user device 106, a wearable device 104, or some combination of components from these devices. The process flow 600 may be implemented by the wearable device diagram 300, timing diagram 400, timing diagram 500, or a combination thereof. Alternative examples of the following may be implemented, where some steps are performed in a different order than described or not performed at all. In some cases, steps may include additional features not mentioned below, or further steps may be added.

At 605, the system may generate reference cardiac pulse waveforms. In some examples, rather than generating reference cardiac pulse waveforms, the system may access previously stored reference cardiac pulse waveforms. The reference cardiac pulse waveforms may be based on a portion of the PPG signal measured by the wearable device before the change in contact. As described herein, the change in contact may be detected by comparing the morphology of previous pulses (e.g., reference cardiac pulse waveforms) to current pulses.

At 610, the system may obtain the PPG signal. For example, the wearable device may measure, by the one or more optical components, the PPG signal for the user of the wearable device. The PPG signal may be associated with a first set of the one or more optical components (e.g., light-emitting components and the optical receivers as described with reference to FIG. 3). In some cases, the user device may receive the PPG signal measured by the wearable device.

At 615, the system may generate the cardiac pulse waveforms. The cardiac pulse waveforms may be generated in response to obtaining the PPG signal. For example, the system may generate the cardiac pulse waveforms from the PPG signal, as described with reference to FIG. 5.

At 620, the system may detect a change. For example, the wearable device may detect a change in the PPG signal. In some cases, the wearable device may detect a change in morphology between cardiac pulse waveforms. The change in the morphology between cardiac pulse waveforms may be based on the PPG signal and reference cardiac pulse waveforms. In some examples, the user device may detect a change in the PPG signal, a change in morphology between cardiac pulse waveforms, or both.

At 625, the system may determine a timing of change in a metric. The second metric may be an example of pulse amplitude, temperature, acceleration, reflected light (e.g., of VCSEL), or a combination thereof. In some examples, the wearable device may determine that the change in the PPG signal or the change in morphology is based on the change in the PPG signal or the change in morphology coinciding with a change in the second metric measured by the wearable device. The change in the second metric may be an example of a change in amplitude between the cardiac pulse waveforms, a change in skin or body temperature of the user, a change in acceleration of the wearable device, a change in light reflected into a laser component, or a combination thereof.

In such cases, the wearable device may determine a change in pulse amplitude, temperature, acceleration, reflected light, or a combination thereof, and in response to the determination, the system may determine the change in the PPG signal or the change in morphology. In some examples, the change in the second metric is measured by a galvanic sensor of the wearable device. The galvanic sensor may be positioned near optical components in the wearable device.

In some cases, the change in contact may be detected based on a threshold change in the PPG DC level. For example, the wearable device may determine that the DC level of the PPG signal has changed by a threshold amount within a threshold duration of time.

At 630, the system may determine change in contact. For example, the wearable device may determine a change in contact between the wearable device and the skin of a user of the wearable device. In some examples, the user device may determine, based on the change in the PPG signal or the change in morphology coinciding with a change in a metric measured by the wearable device, that the change in the PPG signal or the change in morphology is based on a change in contact between the wearable device and the skin of a user of the wearable device. In some examples, the change in contact is detected based on a threshold change in the PPG DC level.

At 635, the system may adjust a calculation for a physiological metric. For example, the wearable device may determine, using at least a portion of the output PPG signal, a physiological metric for the user based on the change in the PPG signal or the change in morphology being based on the change in contact. The wearable device may determine the physiological metric for the user based on the change in contact and the PPG signal. The physiological metric may be an example of blood pressure, blood oxygen level, heart rate, or heart rate variability.

In some cases, the wearable device may output an indication to exclude a portion of the PPG signal from a calculation to determine the physiological metric for the user based on the portion of the PPG signal coinciding with the change in contact. In such cases, PPG data obtained with poor contact between the wearable device and the user may be discarded when calculating the physiological metric. In some examples, the wearable device may output an indication to apply a weighting factor to a portion of the PPG signal based on the portion of the PPG signal coinciding with the change in contact. The PPG data obtained with poor contact between the wearable device and the user may be weighted differently when calculating the physiological metric.

The user device may determine, using at least a portion of the PPG signal, a physiological metric for the user based on the change in the PPG signal or the change in morphology. In some cases, the user device may exclude a portion of the PPG signal from a calculation to determine the physiological metric based on the portion of the PPG signal coinciding with the change in contact. In other examples, the user device may apply a weighting factor to the portion of the PPG signal based on the portion of the PPG signal coinciding with the change in contact. For example, the user device may discard the PPG data obtained from the user when there is poor contact between the wearable device and the user, apply a weighting factor to the PPG data obtained from the user when there is poor contact between the wearable device and the user, or both.

At 640, the system may adjust sensor settings. For example, the wearable device may adjust one or more settings of the one or more optical components of the wearable device based on the change in contact. In such cases, the sensor settings may be adjusted to compensate for the change in contact between the wearable device and the user. The system may deactivate the sensors that have poor contact with the wearable device and activate the sensors that are contacting the wearable device.

At 645, the system may determine a difference between PPG signals. For example, the wearable device may determine, based on the change in contact, a difference in quality between the PPG signal and a second PPG signal associated with a second set of the one or more optical components. In other examples, the user device may determine a difference in quality between the PPG signal and a second PPG signal associated with a second set of optical components. A comparison of signal quality for different channels may inform the user of a re-orientation recommendation for the wearable device.

At 650, the system may prompt an orientation adjustment. For example, the system may generate a user notification for wearable device adjustment and transmit the notification to the user. The user notification may be an example of an alert displayed on the GUI of the user device, a message displayed on the GUI, or both. The alert may indicate insufficient skin contact between the user and the wearable device. The message may indicate “Please adjust the orientation of the wearable device to improve data measurement.”

The wearable device may output an indication of the change in contact to the user device, another device, to another component of the wearable device for processing, or a combination thereof. The wearable device may output the indication to adjust an orientation of the wearable device based on the change in contact. In such cases, the wearable device may indicate that a re-orientation of the wearable device may improve the contact between the wearable device and the user. In some cases, the wearable device may output the PPG signal.

The user device may cause, based on the change in contact, the GUI to display a message prompting the user to adjust an orientation of the wearable device. In such cases, the user may be prompted to re-orient the wearable device. The indication to adjust the orientation (e.g., via the alert, the message, or both) may be based on the difference in quality between the PPG signals. As such, the comparison of signal quality for different channels may inform the user of the re-orientation recommendation for the wearable device.

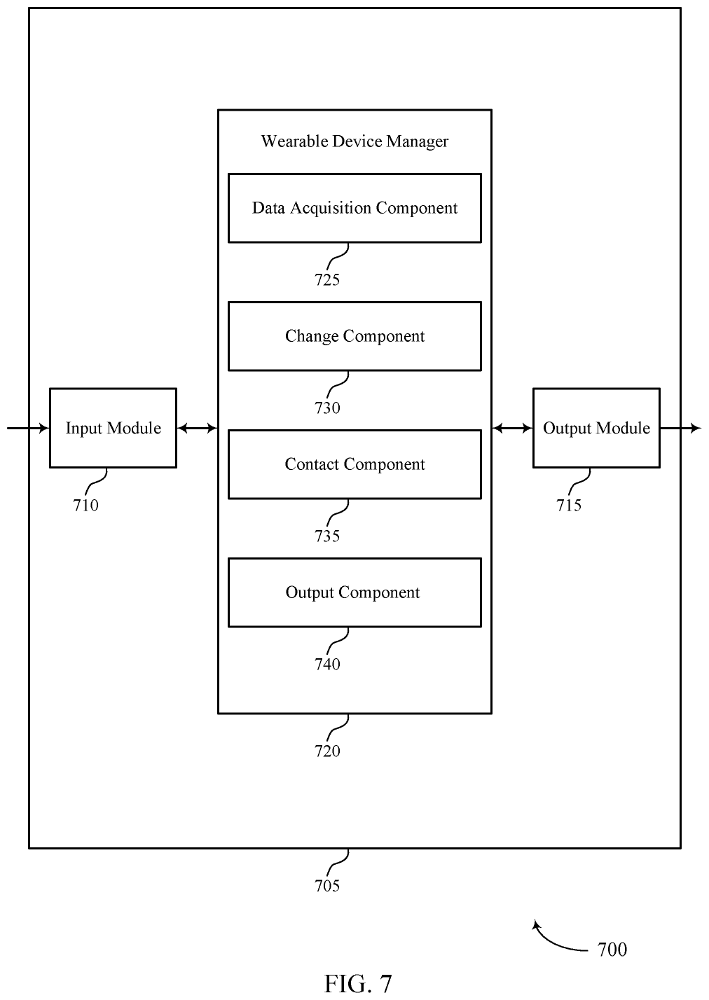

FIG. 7 shows a block diagram 700 of a device 705 that supports contact detection for a wearable device in accordance with aspects of the present disclosure. The device 705 may include an input module 710, an output module 715, and a wearable device manager 720. The device 705 may also include a processor. Each of these components may be in communication with one another (e.g., via one or more buses).

For example, the wearable device manager 720 may include a data acquisition component 725, a change component 730, a contact component 735, an output component 740, an output component 745, or any combination thereof. In some examples, the wearable device manager 720, or various components thereof, may be configured to perform various operations (e.g., receiving, monitoring, transmitting) using or otherwise in cooperation with the input module 710, the output module 715, or both. For example, the wearable device manager 720 may receive information from the input module 710, send information to the output module 715, or be integrated in combination with the input module 710, the output module 715, or both to receive information, transmit information, or perform various other operations as described herein.

The data acquisition component 725 may be configured as or otherwise support a means for measuring, by the one or more optical components, a PPG signal for a user of the wearable device. The change component 730 may be configured as or otherwise support a means for detecting a change in the PPG signal or a change in morphology between cardiac pulse waveforms that are based at least in part on the PPG signal and reference cardiac pulse waveforms. The contact component 735 may be configured as or otherwise support a means for determining, based at least in part on the change in the PPG signal or the change in morphology coinciding with a change in a metric measured by the wearable device, that the change in the PPG signal or the change in morphology is based at least in part on a change in contact between the wearable device and the skin of the user of the wearable device. The output component 740 may be configured as or otherwise support a means for outputting the PPG signal and an indication of the change in contact.

The change component 730 may be configured as or otherwise support a means for detecting a change in a PPG signal measured by a wearable ring device or a change in morphology between cardiac pulse waveforms that are based at least in part on the PPG signal and reference cardiac pulse waveforms. The contact component 735 may be configured as or otherwise support a means for determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device. The output component 740 may be configured as or otherwise support a means for determining, using at least a portion of the PPG signal, a physiological metric for the user based at least in part on the change in the PPG signal or the change in morphology being based at least in part on the change in contact.

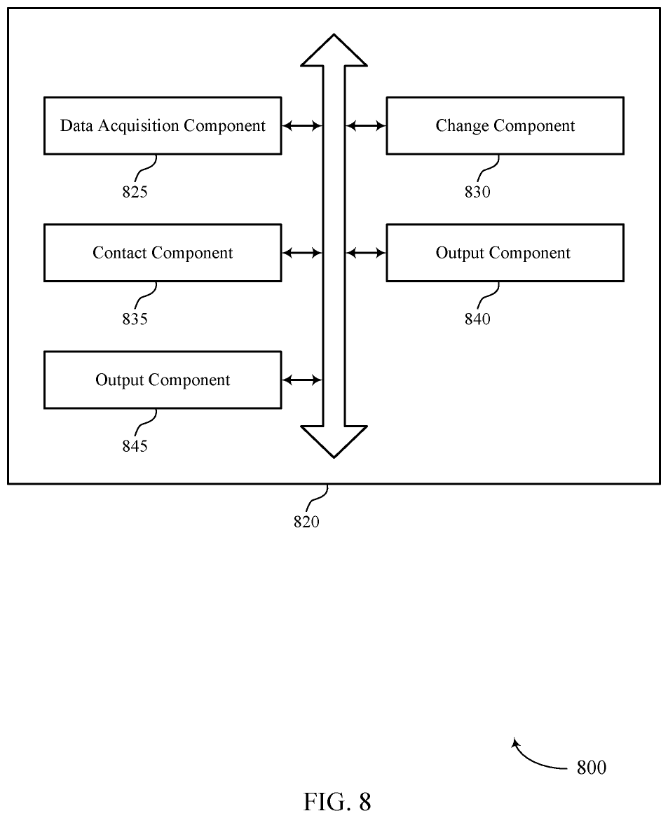

FIG. 8 shows a block diagram 800 of a wearable device manager 820 that supports contact detection for a wearable device in accordance with aspects of the present disclosure. The wearable device manager 820 may be an example of aspects of a wearable device manager or a wearable device manager 720, or both, as described herein. The wearable device manager 820, or various components thereof, may be an example of means for performing various aspects of contact detection for a wearable device as described herein. For example, the wearable device manager 820 may include a data acquisition component 825, a change component 830, a contact component 835, an output component 840, an output component 845, or any combination thereof. Each of these components may communicate, directly or indirectly, with one another (e.g., via one or more buses).

The data acquisition component 825 may be configured as or otherwise support a means for measuring, by the one or more optical components, a PPG signal for a user of the wearable device. The change component 830 may be configured as or otherwise support a means for detecting a change in the PPG signal or a change in morphology between cardiac pulse waveforms that are based at least in part on the PPG signal and reference cardiac pulse waveforms. The contact component 835 may be configured as or otherwise support a means for determining, based at least in part on the change in the PPG signal or the change in morphology coinciding with a change in a metric measured by the wearable device, that the change in the PPG signal or the change in morphology is based at least in part on a change in contact between the wearable device and the skin of the user of the wearable device. The output component 840 may be configured as or otherwise support a means for outputting the PPG signal and an indication of the change in contact.

In some examples, to detect the change in the PPG signal, the change component 830 may be configured as or otherwise support a means for determining that a DC level of the PPG signal has changed by a threshold amount within a threshold duration of time.

In some examples, the reference cardiac pulse waveforms are based at least in part on a portion of the PPG signal measured by the wearable device before the change in contact.

In some examples, the change in the metric comprises a change in amplitude between the cardiac pulse waveforms, a change in skin or body temperature of the user, a change in acceleration of the wearable device, or a change in light reflected into a laser component.

In some examples, the change in the metric is measured by a galvanic sensor of the wearable device.

In some examples, the change component 830 may be configured as or otherwise support a means for determining, using at least a portion of the output PPG signal, a physiological metric for the user based at least in part on the change in the PPG signal or the change in morphology being based at least in part on the change in contact.

In some examples, the physiological metric comprises blood pressure, blood oxygen level, heart rate, or heart rate variability.

In some examples, the output component 840 may be configured as or otherwise support a means for outputting an indication to exclude a portion of the PPG signal from a calculation to determine a physiological metric for the user based at least in part on the portion of the PPG signal coinciding with the change in contact.

In some examples, the output component 840 may be configured as or otherwise support a means for outputting an indication to apply a weighting factor to a portion of the PPG signal based at least in part on the portion of the PPG signal coinciding with the change in contact.

In some examples, the change component 830 may be configured as or otherwise support a means for adjusting one or more settings of the one or more optical components of the wearable device based at least in part on the change in contact.

In some examples, the output component 840 may be configured as or otherwise support a means for outputting an indication to adjust an orientation of the wearable device based at least in part on the change in contact.

In some examples, the PPG signal is associated with a first set of the one or more optical components. In some examples, the change component 830 may be configured as or otherwise support a means for determining, based at least in part on the change in contact, a difference in quality between the PPG signal and a second PPG signal associated with a second set of the one or more optical components, wherein the indication to adjust the orientation is based at least in part on the difference in quality.

In some examples, the change component 830 may be configured as or otherwise support a means for detecting a change in a PPG signal measured by a wearable ring device or a change in morphology between cardiac pulse waveforms that are based at least in part on the PPG signal and reference cardiac pulse waveforms. In some examples, the contact component 835 may be configured as or otherwise support a means for determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device. The output component 840 may be configured as or otherwise support a means for determining, using at least a portion of the PPG signal, a physiological metric for the user based at least in part on the change in the PPG signal or the change in morphology being based at least in part on the change in contact.

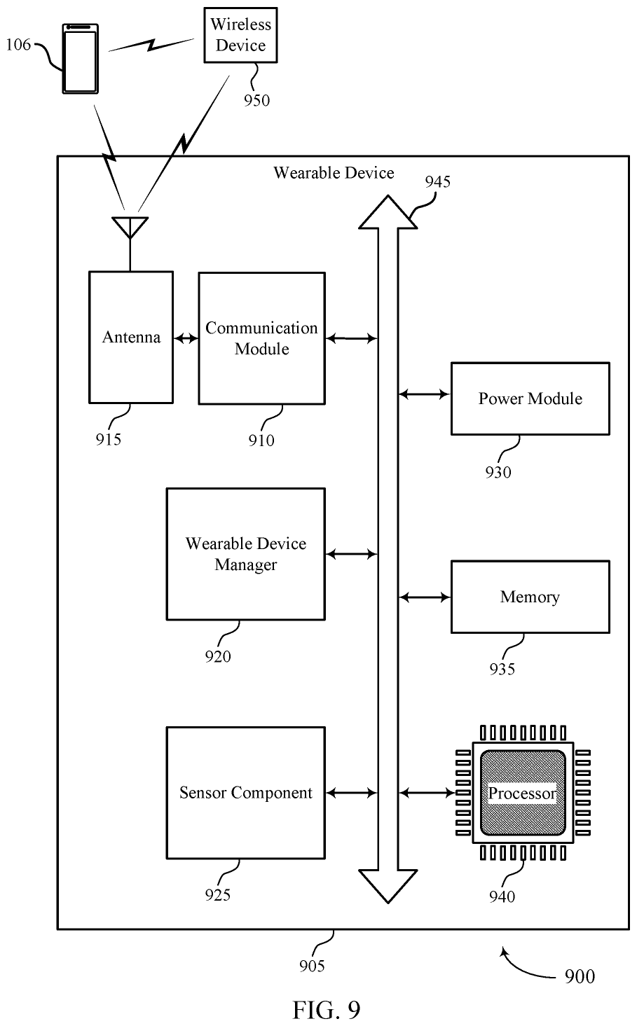

FIG. 9 shows a diagram of a system 900 including a device 905 that supports contact detection for a wearable device in accordance with aspects of the present disclosure. The device 905 may be an example of or include the components of a device 705 as described herein. The device 905 may include an example of a wearable device 104, as described previously herein. The device 905 may include components for bi-directional communications including components for transmitting and receiving communications with a user device 106 and a server 110, such as a wearable device manager 920, a communication module 910, an antenna 915, a sensor component 925, a power module 930, a memory 935, a processor 940, and a wireless device 950. These components may be in electronic communication or otherwise coupled (e.g., operatively, communicatively, functionally, electronically, electrically) via one or more buses (e.g., a bus 945).

For example, the wearable device manager 920 may be configured as or otherwise support a means for measuring, by the one or more optical components, a PPG signal for a user of the wearable device. The wearable device manager 920 may be configured as or otherwise support a means for detecting a change in the PPG signal or a change in morphology between cardiac pulse waveforms that are based at least in part on the PPG signal and reference cardiac pulse waveforms. The wearable device manager 920 may be configured as or otherwise support a means for determining, based at least in part on the change in the PPG signal or the change in morphology coinciding with a change in a metric measured by the wearable device, that the change in the PPG signal or the change in morphology is based at least in part on a change in contact between the wearable device and the skin of the user of the wearable device. The wearable device manager 920 may be configured as or otherwise support a means for outputting the PPG signal and an indication of the change in contact.

For example, the wearable device manager 920 may be configured as or otherwise support a means for detecting a change in a PPG signal measured by a wearable ring device or a change in morphology between cardiac pulse waveforms that are based at least in part on the PPG signal and reference cardiac pulse waveforms. The wearable device manager 920 may be configured as or otherwise support a means for determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device. The wearable device manager 920 may be configured as or otherwise support a means for determining, using at least a portion of the PPG signal, a physiological metric for the user based at least in part on the change in the PPG signal or the change in morphology being based at least in part on the change in contact.

By including or configuring the wearable device manager 920 in accordance with examples as described herein, the device 905 may support techniques for improved communication reliability, reduced latency, improved user experience related to reduced processing, reduced power consumption, more efficient utilization of communication resources, improved coordination between devices, longer battery life, improved utilization of processing capability, and the like.

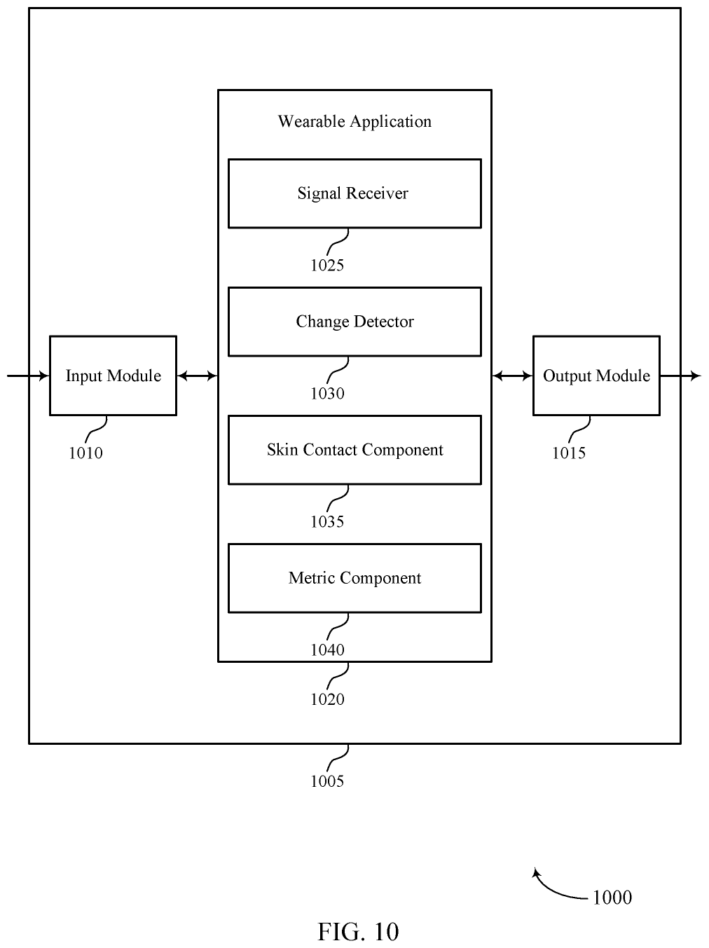

FIG. 10 shows a block diagram 1000 of a device 1005 that supports contact detection for a wearable device in accordance with aspects of the present disclosure. The device 1005 may include an input module 1010, an output module 1015, and a wearable application 1020. The device 1005 may also include a processor. Each of these components may be in communication with one another (e.g., via one or more buses).

The input module 1010 may provide a means for receiving information such as packets, user data, control information, or any combination thereof associated with various information channels (e.g., control channels, data channels, information channels related to illness detection techniques). Information may be passed on to other components of the device 1005. The input module 1010 may utilize a single antenna or a set of multiple antennas.

The output module 1015 may provide a means for transmitting signals generated by other components of the device 1005. For example, the output module 1015 may transmit information such as packets, user data, control information, or any combination thereof associated with various information channels (e.g., control channels, data channels, information channels related to illness detection techniques). In some examples, the output module 1015 may be co-located with the input module 1010 in a transceiver module. The output module 1015 may utilize a single antenna or a set of multiple antennas.

For example, the wearable application 1020 may include a signal receiver 1025, a change detector 1030, a skin contact component 1035, a metric component 1040, or any combination thereof. In some examples, the wearable application 1020, or various components thereof, may be configured to perform various operations (e.g., receiving, monitoring, transmitting) using or otherwise in cooperation with the input module 1010, the output module 1015, or both. For example, the wearable application 1020 may receive information from the input module 1010, send information to the output module 1015, or be integrated in combination with the input module 1010, the output module 1015, or both to receive information, transmit information, or perform various other operations as described herein.

The signal receiver 1025 may be configured as or otherwise support a means for receiving a PPG signal measured by the wearable device. The change detector 1030 may be configured as or otherwise support a means for detecting a change in the PPG signal or a change in morphology between cardiac pulse waveforms that are based at least in part on the PPG signal and reference cardiac pulse waveforms. The skin contact component 1035 may be configured as or otherwise support a means for determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device. The metric component 1040 may be configured as or otherwise support a means for determining, using at least a portion of the PPG signal, a physiological metric for the user based at least in part on the change in the PPG signal or the change in morphology being based at least in part on the change in contact.

In some examples, the change detector 1030 may be configured as or otherwise support a means for detecting a change in a PPG signal measured by a wearable ring device or a change in morphology between cardiac pulse waveforms that are based at least in part on the PPG signal and reference cardiac pulse waveforms. The skin contact component 1035 may be configured as or otherwise support a means for determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device. The metric component 1045 may be configured as or otherwise support a means for determining, using at least a portion of the PPG signal, a physiological metric for the user based at least in part on the change in the PPG signal or the change in morphology being based at least in part on the change in contact.

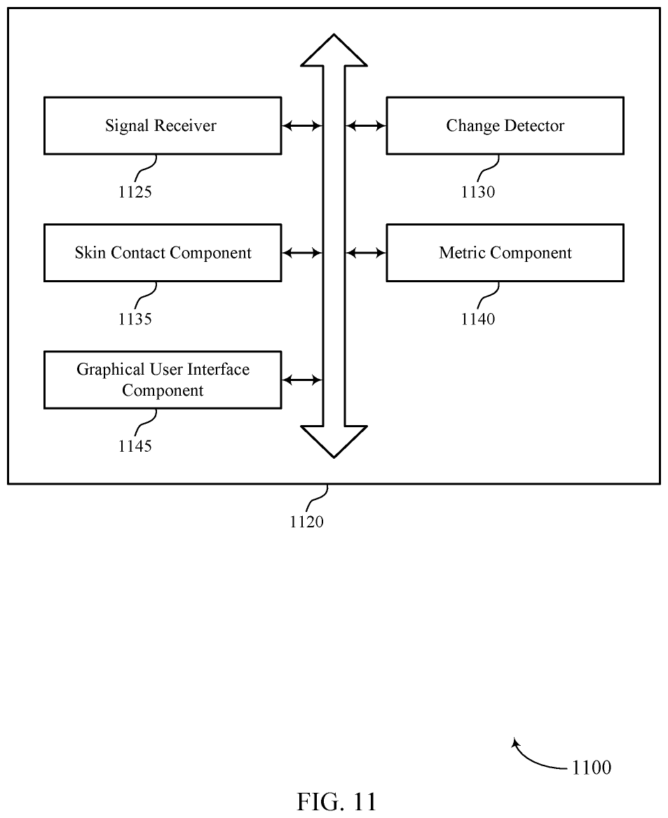

FIG. 11 shows a block diagram 1100 of a wearable application 1120 that supports contact detection for a wearable device in accordance with aspects of the present disclosure. The wearable application 1120 may be an example of aspects of a wearable application or a wearable application 1020, or both, as described herein. The wearable application 1120, or various components thereof, may be an example of means for performing various aspects of contact detection for a wearable device as described herein. For example, the wearable application 1120 may include a signal receiver 1125, a change detector 1130, a skin contact component 1135, a metric component 1140, a graphical user interface component 1145, or any combination thereof. Each of these components may communicate, directly or indirectly, with one another (e.g., via one or more buses).

The signal receiver 1125 may be configured as or otherwise support a means for receiving a PPG signal measured by the wearable device. The change detector 1130 may be configured as or otherwise support a means for detecting a change in the PPG signal or a change in morphology between cardiac pulse waveforms that are based at least in part on the PPG signal and reference cardiac pulse waveforms. The skin contact component 1135 may be configured as or otherwise support a means for determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device. The metric component 1140 may be configured as or otherwise support a means for determining, using at least a portion of the PPG signal, a physiological metric for the user based at least in part on the change in the PPG signal or the change in morphology being based at least in part on the change in contact.

In some examples, to detect the change in the PPG signal, the change detector 1130 may be configured as or otherwise support a means for determining that a DC level of the PPG signal has changed by a threshold amount within a threshold duration of time.

In some examples, the reference cardiac pulse waveforms are based at least in part on a portion of the PPG signal measured by the wearable device before the change in contact.

In some examples, the change in the metric comprises a change in amplitude between the cardiac pulse waveforms, a change in skin or body temperature of the user, a change in acceleration of the wearable device, or a change in light reflected into a laser component.

In some examples, to determine the physiological metric, the metric component 1140 may be configured as or otherwise support a means for excluding a second portion of the PPG signal from a calculation to determine the physiological metric based at least in part on the second portion of the PPG signal coinciding with the change in contact.

In some examples, to determine the physiological metric, the metric component 1140 may be configured as or otherwise support a means for applying a weighting factor to a second portion of the PPG signal based at least in part on the second portion of the PPG signal coinciding with the change in contact.

In some examples, the graphical user interface component 1145 may be configured as or otherwise support a means for causing, based at least in part on the change in contact, a graphical user interface to display a message prompting the user to adjust an orientation of the wearable device.

In some examples, the PPG signal is associated with a first set of optical components. In some examples, the change detector 1130 may be configured as or otherwise support a means for determining a difference in quality between the PPG signal and a second PPG signal associated with a second set of optical components, wherein the message to adjust the orientation is based at least in part on the difference in quality.

The change detector 1130 may be configured as or otherwise support a means for detecting a change in a PPG signal measured by a wearable ring device or a change in morphology between cardiac pulse waveforms that are based at least in part on the PPG signal and reference cardiac pulse waveforms. The skin contact component 1135 may be configured as or otherwise support a means for determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device. The metric component 1140 may be configured as or otherwise support a means for determining, using at least a portion of the PPG signal, a physiological metric for the user based at least in part on the change in the PPG signal or the change in morphology being based at least in part on the change in contact.

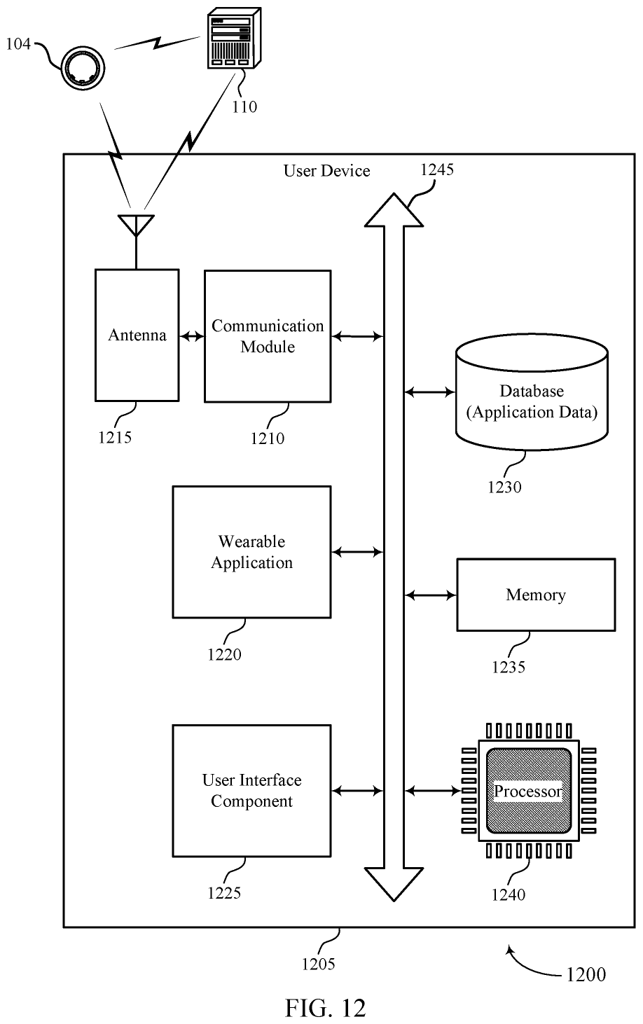

FIG. 12 shows a diagram of a system 1200 including a device 1205 that supports contact detection for a wearable device in accordance with aspects of the present disclosure. The device 1205 may be an example of or include the components of a device 1005 as described herein. The device 1205 may include an example of a user device 106, as described previously herein. The device 1205 may include components for bi-directional communications including components for transmitting and receiving communications with a wearable device 104 and a server 110, such as a wearable application 1220, a communication module 1210, an antenna 1215, a user interface component 1225, a database (application data) 1230, a memory 1235, and a processor 1240. These components may be in electronic communication or otherwise coupled (e.g., operatively, communicatively, functionally, electronically, electrically) via one or more buses (e.g., a bus 1245).

The communication module 1210 may manage input and output signals for the device 1205 via the antenna 1215. The communication module 1210 may include an example of the communication module 220-b of the user device 106 shown and described in FIG. 2. In this regard, the communication module 1210 may manage communications with the ring 104 and the server 110, as illustrated in FIG. 2. The communication module 1210 may also manage peripherals not integrated into the device 1205. In some cases, the communication module 1210 may represent a physical connection or port to an external peripheral. In some cases, the communication module 1210 may utilize an operating system such as iOS®, ANDROID®, MS-DOS®, MS-WINDOWS®, OS/2®, UNIX®, LINUX®, or another known operating system. In other cases, the communication module 1210 may represent or interact with a wearable device (e.g., ring 104), modem, a keyboard, a mouse, a touchscreen, or a similar device. In some cases, the communication module 1210 may be implemented as part of the processor 1240. In some examples, a user may interact with the device 1205 via the communication module 1210, user interface component 1225, or via hardware components controlled by the communication module 1210.

In some cases, the device 1205 may include a single antenna 1215. However, in some other cases, the device 1205 may have more than one antenna 1215, which may be capable of concurrently transmitting or receiving multiple wireless transmissions. The communication module 1210 may communicate bi-directionally, via the one or more antennas 1215, wired, or wireless links as described herein. For example, the communication module 1210 may represent a wireless transceiver and may communicate bi-directionally with another wireless transceiver. The communication module 1210 may also include a modem to modulate the packets, to provide the modulated packets to one or more antennas 1215 for transmission, and to demodulate packets received from the one or more antennas 1215.

The user interface component 1225 may manage data storage and processing in a database 1230. In some cases, a user may interact with the user interface component 1225. In other cases, the user interface component 1225 may operate automatically without user interaction. The database 1230 may be an example of a single database, a distributed database, multiple distributed databases, a data store, a data lake, or an emergency backup database.

The memory 1235 may include RAM and ROM. The memory 1235 may store computer-readable, computer-executable software including instructions that, when executed, cause the processor 1240 to perform various functions described herein. In some cases, the memory 1235 may contain, among other things, a BIOS which may control basic hardware or software operation such as the interaction with peripheral components or devices.

The processor 1240 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 1240 may be configured to operate a memory array using a memory controller. In other cases, a memory controller may be integrated into the processor 1240. The processor 1240 may be configured to execute computer-readable instructions stored in a memory 1235 to perform various functions (e.g., functions or tasks supporting a method and system for sleep staging algorithms).

For example, the wearable application 1220 may be configured as or otherwise support a means for receiving a PPG signal measured by the wearable device. The wearable application 1220 may be configured as or otherwise support a means for detecting a change in the PPG signal or a change in morphology between cardiac pulse waveforms that are based at least in part on the PPG signal and reference cardiac pulse waveforms. The wearable application 1220 may be configured as or otherwise support a means for determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device. The wearable application 1220 may be configured as or otherwise support a means for determining, using at least a portion of the PPG signal, a physiological metric for the user based at least in part on the change in the PPG signal or the change in morphology being based at least in part on the change in contact.

For example, the wearable application 1220 may be configured as or otherwise support a means for detecting a change in the PPG signal measured by a wearable ring device or a change in morphology between cardiac pulse waveforms that are based at least in part on the PPG signal and reference cardiac pulse waveforms. The wearable application 1220 may be configured as or otherwise support a means for determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device. The wearable application 1220 may be configured as or otherwise support a means for determining, using at least a portion of the PPG signal, a physiological metric for the user based at least in part on the change in the PPG signal or the change in morphology being based at least in part on the change in contact.

By including or configuring the wearable application 1220 in accordance with examples as described herein, the device 1205 may support techniques for improved communication reliability, reduced latency, improved user experience related to reduced processing, reduced power consumption, more efficient utilization of communication resources, improved coordination between devices, longer battery life, improved utilization of processing capability, and the like.

The wearable application 1220 may include an application (e.g., “app”), program, software, or other component which is configured to facilitate communications with a ring 104, server 110, other user devices 106, and the like. For example, the wearable application 1220 may include an application executable on a user device 106 which is configured to receive data (e.g., physiological data) from a ring 104, perform processing operations on the received data, transmit and receive data with the servers 110, and cause presentation of data to a user 102.

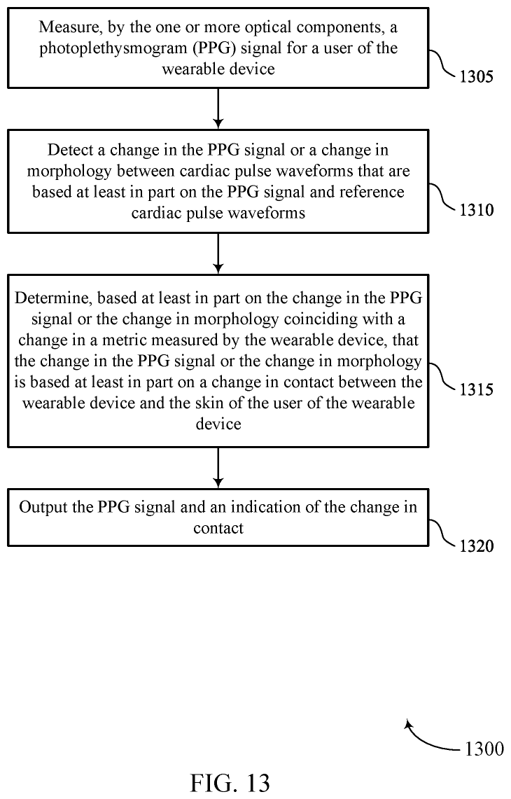

FIG. 13 shows a flowchart illustrating a method 1300 that supports contact detection for a wearable device in accordance with aspects of the present disclosure. The operations of the method 1300 may be implemented by a wearable device or its components as described herein. For example, the operations of the method 1300 may be performed by a wearable device as described with reference to FIGS. 1 through 9. In some examples, a wearable device may execute a set of instructions to control the functional elements of the wearable device to perform the described functions. Additionally, or alternatively, the wearable device may perform aspects of the described functions using special-purpose hardware.

At 1305, the method may include measuring, by the one or more optical components, a photoplethysmogram (PPG) signal for a user of the wearable device. The operations of block 1305 may be performed in accordance with examples as disclosed herein. In some examples, aspects of the operations of 1305 may be performed by a data acquisition component 825 as described with reference to FIG. 8.

At 1310, the method may include detecting a change in the PPG signal or a change in morphology between cardiac pulse waveforms that are based at least in part on the PPG signal and reference cardiac pulse waveforms. The operations of block 1310 may be performed in accordance with examples as disclosed herein. In some examples, aspects of the operations of 1310 may be performed by a change component 830 as described with reference to FIG. 8.

At 1315, the method may include determining, based at least in part on the change in the PPG signal or the change in morphology coinciding with a change in a metric measured by the wearable device, that the change in the PPG signal or the change in morphology is based at least in part on a change in contact between the wearable device and the skin of the user of the wearable device. The operations of block 1315 may be performed in accordance with examples as disclosed herein. In some examples, aspects of the operations of 1315 may be performed by a contact component 835 as described with reference to FIG. 8.

At 1320, the method may include outputting the PPG signal and an indication of the change in contact. The operations of block 1320 may be performed in accordance with examples as disclosed herein. In some examples, aspects of the operations of 1320 may be performed by an output component 840 as described with reference to FIG. 8.

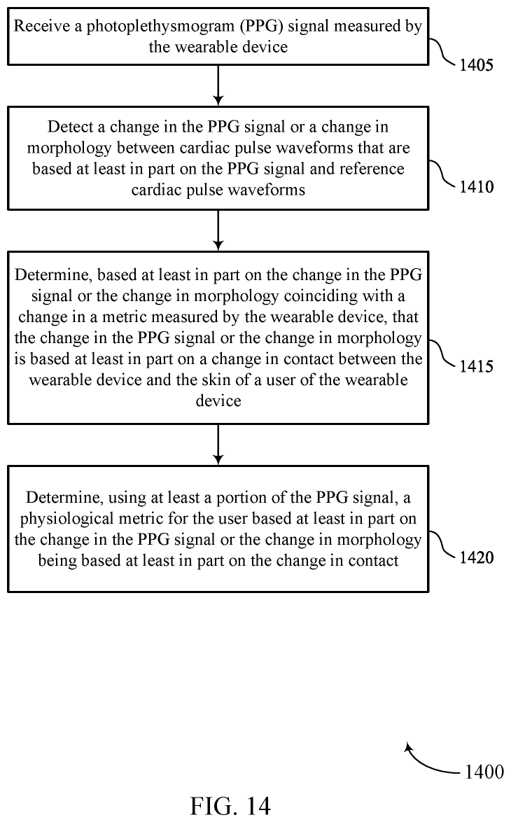

FIG. 14 shows a flowchart illustrating a method 1400 that supports contact detection for a wearable device in accordance with aspects of the present disclosure. The operations of the method 1400 may be implemented by a user device or its components as described herein. For example, the operations of the method 1400 may be performed by a user device as described with reference to FIGS. 1 through 6 and 10 through 12. In some examples, a user device may execute a set of instructions to control the functional elements of the user device to perform the described functions. Additionally, or alternatively, the user device may perform aspects of the described functions using special-purpose hardware.

At 1405, the method may include receiving a photoplethysmogram (PPG) signal measured by the wearable device. The operations of block 1405 may be performed in accordance with examples as disclosed herein. In some examples, aspects of the operations of 1405 may be performed by a signal receiver 1125 as described with reference to FIG. 11.

At 1410, the method may include detecting a change in the PPG signal or a change in morphology between cardiac pulse waveforms that are based at least in part on the PPG signal and reference cardiac pulse waveforms. The operations of block 1410 may be performed in accordance with examples as disclosed herein. In some examples, aspects of the operations of 1410 may be performed by a change detector 1130 as described with reference to FIG. 11.

At 1415, the method may include determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device. The operations of block 1415 may be performed in accordance with examples as disclosed herein. In some examples, aspects of the operations of 1415 may be performed by a skin contact component 1135 as described with reference to FIG. 11.

At 1420, the method may include determining, using at least a portion of the PPG signal, a physiological metric for the user based at least in part on the change in the PPG signal or the change in morphology being based at least in part on the change in contact. The operations of block 1420 may be performed in accordance with examples as disclosed herein. In some examples, aspects of the operations of 1420 may be performed by a metric component 1140 as described with reference to FIG. 11.

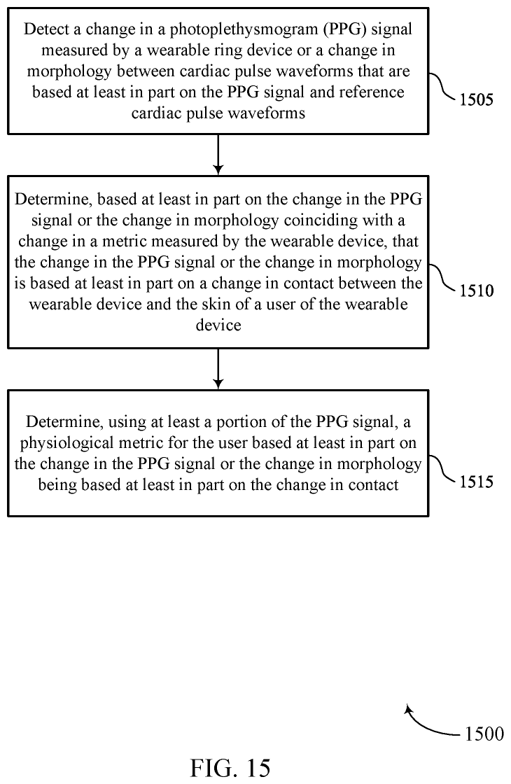

FIG. 15 shows a flowchart illustrating a method 1500 that supports contact detection for a wearable device in accordance with aspects of the present disclosure. The operations of the method 1500 may be implemented by a wearable device or its components as described herein. For example, the operations of the method 1500 may be performed by a wearable device as described with reference to FIGS. 1 through 9 or a user device as described with reference to FIGS. 1 through 6 and 10 through 12. In some examples, a wearable device or a user device may execute a set of instructions to control the functional elements of the wearable device or user device, respectively, to perform the described functions. Additionally, or alternatively, the wearable device or user device may perform aspects of the described functions using special-purpose hardware.

At 1505, the method may include detecting a change in a photoplethysmogram (PPG) signal measured by a wearable ring device or a change in morphology between cardiac pulse waveforms that are based at least in part on the PPG signal and reference cardiac pulse waveforms. The operations of block 1505 may be performed in accordance with examples as disclosed herein. In some examples, aspects of the operations of 1505 may be performed by a change component 830 as described with reference to FIG. 8.

At 1510, the method may include determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device. The operations of block 1510 may be performed in accordance with examples as disclosed herein. In some examples, aspects of the operations of 1510 may be performed by a contact component 835 as described with reference to FIG. 8.

At 1515, the method may include determining, using at least a portion of the PPG signal, a physiological metric for the user based at least in part on the change in the PPG signal or the change in morphology being based at least in part on the change in contact. The operations of block 1515 may be performed in accordance with examples as disclosed herein. In some examples, aspects of the operations of 1515 may be performed by an output component 845 as described with reference to FIG. 8.

It should be noted that the methods described above describe possible implementations, and that the operations and the steps may be rearranged or otherwise modified and that other implementations are possible. Furthermore, aspects from two or more of the methods may be combined.

A method is described. The method may include measuring, by the one or more optical components, a PPG signal for a user of the wearable device, detecting a change in the PPG signal or a change in morphology between cardiac pulse waveforms that are based at least in part on the PPG signal and reference cardiac pulse waveforms, determining, based at least in part on the change in the PPG signal or the change in morphology coinciding with a change in a metric measured by the wearable device, that the change in the PPG signal or the change in morphology is based at least in part on a change in contact between the wearable device and the skin of the user of the wearable device, and outputting the PPG signal and an indication of the change in contact.

An apparatus is described. The apparatus may include a processor, memory coupled with the processor, and instructions stored in the memory. The instructions may be executable by the processor to cause the apparatus to measure, by the one or more optical components, a PPG signal for a user of the wearable device, detect a change in the PPG signal or a change in morphology between cardiac pulse waveforms that are based at least in part on the PPG signal and reference cardiac pulse waveforms, determine, based at least in part on the change in the PPG signal or the change in morphology coinciding with a change in a metric measured by the wearable device, that the change in the PPG signal or the change in morphology is based at least in part on a change in contact between the wearable device and the skin of the user of the wearable device, and output the PPG signal and an indication of the change in contact.

Another apparatus is described. The apparatus may include means for measuring, by the one or more optical components, a PPG signal for a user of the wearable device, means for detecting a change in the PPG signal or a change in morphology between cardiac pulse waveforms that are based at least in part on the PPG signal and reference cardiac pulse waveforms, means for determining, based at least in part on the change in the PPG signal or the change in morphology coinciding with a change in a metric measured by the wearable device, that the change in the PPG signal or the change in morphology is based at least in part on a change in contact between the wearable device and the skin of the user of the wearable device, and means for outputting the PPG signal and an indication of the change in contact.

A non-transitory computer-readable medium storing code is described. The code may include instructions executable by a processor to measure, by the one or more optical components, a PPG signal for a user of the wearable device, detect a change in the PPG signal or a change in morphology between cardiac pulse waveforms that are based at least in part on the PPG signal and reference cardiac pulse waveforms, determine, based at least in part on the change in the PPG signal or the change in morphology coinciding with a change in a metric measured by the wearable device, that the change in the PPG signal or the change in morphology is based at least in part on a change in contact between the wearable device and the skin of the user of the wearable device, and output the PPG signal and an indication of the change in contact.

A wearable device is described. The wearable device may include one or more optical components, a memory, and a processor, the wearable device configured to measure, by the one or more optical components, a PPG signal for a user of the wearable device, detect a change in the PPG signal or a change in morphology between cardiac pulse waveforms that are based at least in part on the PPG signal and reference cardiac pulse waveforms, determine, based at least in part on the change in the PPG signal or the change in morphology coinciding with a change in a metric measured by the wearable device, that the change in the PPG signal or the change in morphology is based at least in part on a change in contact between the wearable device and the skin of the user of the wearable device, and output the PPG signal and an indication of the change in contact.

Some examples of the method, apparatuses, wearable device, and non-transitory computer-readable medium described herein may further include operations, features, means, or instructions for determining that a DC level of the PPG signal may have changed by a threshold amount within a threshold duration of time.

In some examples of the method, apparatuses, wearable device, and non-transitory computer-readable medium described herein, the reference cardiac pulse waveforms may be based at least in part on a portion of the PPG signal measured by the wearable device before the change in contact.

In some examples of the method, apparatuses, wearable device, and non-transitory computer-readable medium described herein, the change in the metric comprises a change in amplitude between the cardiac pulse waveforms, a change in skin or body temperature of the user, a change in acceleration of the wearable device, or a change in light reflected into a laser component.

In some examples of the method, apparatuses, wearable device, and non-transitory computer-readable medium described herein, the change in the metric may be measured by a galvanic sensor of the wearable device.

Some examples of the method, apparatuses, wearable device, and non-transitory computer-readable medium described herein may further include operations, features, means, or instructions for determining, using at least a portion of the output PPG signal, a physiological metric for the user based at least in part on the change in the PPG signal or the change in morphology being based at least in part on the change in contact.

In some examples of the method, apparatuses, wearable device, and non-transitory computer-readable medium described herein, the physiological metric comprises blood pressure, blood oxygen level, heart rate, or heart rate variability.

Some examples of the method, apparatuses, wearable device, and non-transitory computer-readable medium described herein may further include operations, features, means, or instructions for outputting an indication to exclude a portion of the PPG signal from a calculation to determine a physiological metric for the user based at least in part on the portion of the PPG signal coinciding with the change in contact.

Some examples of the method, apparatuses, wearable device, and non-transitory computer-readable medium described herein may further include operations, features, means, or instructions for outputting an indication to apply a weighting factor to a portion of the PPG signal based at least in part on the portion of the PPG signal coinciding with the change in contact.

Some examples of the method, apparatuses, wearable device, and non-transitory computer-readable medium described herein may further include operations, features, means, or instructions for adjusting one or more settings of the one or more optical components of the wearable device based at least in part on the change in contact.

Some examples of the method, apparatuses, wearable device, and non-transitory computer-readable medium described herein may further include operations, features, means, or instructions for outputting an indication to adjust an orientation of the wearable device based at least in part on the change in contact.

In some examples of the method, apparatuses, wearable device, and non-transitory computer-readable medium described herein, the PPG signal is associated with a first set of the one or more optical components. Some examples of the method, apparatuses, wearable device, and non-transitory computer-readable medium described herein may further include operations, features, means, or instructions for determining, based at least in part on the change in contact, a difference in quality between the PPG signal and a second PPG signal associated with a second set of the one or more optical components, wherein the indication to adjust the orientation may be based at least in part on the difference in quality.

A method is described. The method may include receiving a PPG signal measured by the wearable device, detecting a change in the PPG signal or a change in morphology between cardiac pulse waveforms that are based at least in part on the PPG signal and reference cardiac pulse waveforms, determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device, and determining, using at least a portion of the PPG signal, a physiological metric for the user based at least in part on the change in the PPG signal or the change in morphology being based at least in part on the change in contact.

An apparatus is described. The apparatus may include a processor, memory coupled with the processor, and instructions stored in the memory. The instructions may be executable by the processor to cause the apparatus to receive a PPG signal measured by the wearable device, detect a change in the PPG signal or a change in morphology between cardiac pulse waveforms that are based at least in part on the PPG signal and reference cardiac pulse waveforms, determine, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device, and determine, using at least a portion of the PPG signal, a physiological metric for the user based at least in part on the change in the PPG signal or the change in morphology being based at least in part on the change in contact.

Another apparatus is described. The apparatus may include means for receiving a PPG signal measured by the wearable device, means for detecting a change in the PPG signal or a change in morphology between cardiac pulse waveforms that are based at least in part on the PPG signal and reference cardiac pulse waveforms, means for determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device, and means for determining, using at least a portion of the PPG signal, a physiological metric for the user based at least in part on the change in the PPG signal or the change in morphology being based at least in part on the change in contact.

A non-transitory computer-readable medium storing code is described. The code may include instructions executable by a processor to receive a PPG signal measured by the wearable device, detect a change in the PPG signal or a change in morphology between cardiac pulse waveforms that are based at least in part on the PPG signal and reference cardiac pulse waveforms, determine, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device, and determine, using at least a portion of the PPG signal, a physiological metric for the user based at least in part on the change in the PPG signal or the change in morphology being based at least in part on the change in contact.

A system is described. The system may include a wearable device including one or more optical components, a memory, and a processor and a user device electronically coupled with the wearable device and configured to receive a PPG signal measured by the wearable device, detect a change in the PPG signal or a change in morphology between cardiac pulse waveforms that are based at least in part on the PPG signal and reference cardiac pulse waveforms, determine, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device, and determine, using at least a portion of the PPG signal, a physiological metric for the user based at least in part on the change in the PPG signal or the change in morphology being based at least in part on the change in contact.

Some examples of the method, apparatuses, system, and non-transitory computer-readable medium described herein may further include operations, features, means, or instructions for determining that a DC level of the PPG signal may have changed by a threshold amount within a threshold duration of time.

In some examples of the method, apparatuses, system, and non-transitory computer-readable medium described herein, the reference cardiac pulse waveforms may be based at least in part on a portion of the PPG signal measured by the wearable device before the change in contact.

In some examples of the method, apparatuses, system, and non-transitory computer-readable medium described herein, the change in the metric comprises a change in amplitude between the cardiac pulse waveforms, a change in skin or body temperature of the user, a change in acceleration of the wearable device, or a change in light reflected into a laser component.

Some examples of the method, apparatuses, system, and non-transitory computer-readable medium described herein may further include operations, features, means, or instructions for excluding a second portion of the PPG signal from a calculation to determine the physiological metric based at least in part on the second portion of the PPG signal coinciding with the change in contact.

Some examples of the method, apparatuses, system, and non-transitory computer-readable medium described herein may further include operations, features, means, or instructions for applying a weighting factor to a second portion of the PPG signal based at least in part on the second portion of the PPG signal coinciding with the change in contact.

Some examples of the method, apparatuses, system, and non-transitory computer-readable medium described herein may further include operations, features, means, or instructions for causing, based at least in part on the change in contact, a graphical user interface to display a message prompting the user to adjust an orientation of the wearable device.

In some examples of the method, apparatuses, system, and non-transitory computer-readable medium described herein, the PPG signal is associated with a first set of optical components. Some examples of the method, apparatuses, system, and non-transitory computer-readable medium described herein may further include operations, features, means, or instructions for determining a difference in quality between the PPG signal and a second PPG signal associated with a second set of optical components, wherein the message to adjust the orientation may be based at least in part on the difference in quality.

A method is described. The method may include detecting a change in a PPG signal measured by a wearable ring device or a change in morphology between cardiac pulse waveforms that are based at least in part on the PPG signal and reference cardiac pulse waveforms, determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device, and determining, using at least a portion of the PPG signal, a physiological metric for the user based at least in part on the change in the PPG signal or the change in morphology being based at least in part on the change in contact.

An apparatus is described. The apparatus may include a processor, memory coupled with the processor, and instructions stored in the memory. The instructions may be executable by the processor to cause the apparatus to detect a change in a PPG signal measured by a wearable ring device or a change in morphology between cardiac pulse waveforms that are based at least in part on the PPG signal and reference cardiac pulse waveforms, determine, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device, and determine, using at least a portion of the PPG signal, a physiological metric for the user based at least in part on the change in the PPG signal or the change in morphology being based at least in part on the change in contact.

Another apparatus is described. The apparatus may include means for detecting a change in a PPG signal measured by a wearable ring device or a change in morphology between cardiac pulse waveforms that are based at least in part on the PPG signal and reference cardiac pulse waveforms, means for determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device, and means for determining, using at least a portion of the PPG signal, a physiological metric for the user based at least in part on the change in the PPG signal or the change in morphology being based at least in part on the change in contact.

A non-transitory computer-readable medium storing code is described. The code may include instructions executable by a processor to detect a change in a PPG signal measured by a wearable ring device or a change in morphology between cardiac pulse waveforms that are based at least in part on the PPG signal and reference cardiac pulse waveforms, determine, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device, and determine, using at least a portion of the PPG signal, a physiological metric for the user based at least in part on the change in the PPG signal or the change in morphology being based at least in part on the change in contact.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable device, comprising:
one or more optical components, a memory, and a processor, the wearable device configured to:
measure, by the one or more optical components, a photoplethysmogram (PPG) signal for a user of the wearable device;
detect a change in the PPG signal or a change in morphology between cardiac pulse waveforms that are based at least in part on the PPG signal and reference cardiac pulse waveforms;
determine, based at least in part on the change in the PPG signal or the change in morphology coinciding with a change in a metric measured by the wearable device, that the change in the PPG signal or the change in morphology is based at least in part on a change in contact between the wearable device and the skin of the user of the wearable device; and
output the PPG signal and an indication of the change in contact.

2. The wearable device of claim 1, wherein the wearable device is configured to detect the change in the PPG signal by being configured to:
determine that a direct current (DC) level of the PPG signal has changed by a threshold amount within a threshold duration of time.

3. The wearable device of claim 1, wherein the reference cardiac pulse waveforms are based at least in part on a portion of the PPG signal measured by the wearable device before the change in contact.

4. The wearable device of claim 1, wherein the change in the metric comprises a change in amplitude between the cardiac pulse waveforms, a change in skin or body temperature of the user, a change in acceleration of the wearable device, or a change in light reflected into a laser component.

5. The wearable device of claim 1, wherein the change in the metric is measured by a galvanic sensor of the wearable device.

6. The wearable device of claim 1, wherein the wearable device is further configured to:
determine, using at least a portion of the output PPG signal, a physiological metric for the user based at least in part on the change in the PPG signal or the change in morphology being based at least in part on the change in contact.

7. The wearable device of claim 6, wherein the physiological metric comprises blood pressure, blood oxygen level, heart rate, or heart rate variability.

8. The wearable device of claim 1, wherein the wearable device is further configured to:
output an indication to exclude a portion of the PPG signal from a calculation to determine a physiological metric for the user based at least in part on the portion of the PPG signal coinciding with the change in contact.

9. The wearable device of claim 1, wherein the wearable device is further configured to:
output an indication to apply a weighting factor to a portion of the PPG signal based at least in part on the portion of the PPG signal coinciding with the change in contact.

10. The wearable device of claim 1, wherein the wearable device is further configured to:
adjust one or more settings of the one or more optical components of the wearable device based at least in part on the change in contact.

11. The wearable device of claim 1, wherein the wearable device is further configured to:
output an indication to adjust an orientation of the wearable device based at least in part on the change in contact.

12. The wearable device of claim 11, wherein the PPG signal is associated with a first set of the one or more optical components, and wherein the wearable device is further configured to:
determine, based at least in part on the change in contact, a difference in quality between the PPG signal and a second PPG signal associated with a second set of the one or more optical components, wherein the indication to adjust the orientation is based at least in part on the difference in quality.

13. A system, comprising:
a wearable device comprising one or more optical components, a memory, and a processor; and
a user device electronically coupled with the wearable device and configured to:
receive a photoplethysmogram (PPG) signal measured by the wearable device;
detect a change in the PPG signal or a change in morphology between cardiac pulse waveforms that are based at least in part on the PPG signal and reference cardiac pulse waveforms;
determine, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device; and
determine, using at least a portion of the PPG signal, a physiological metric for the user based at least in part on the change in the PPG signal or the change in morphology being based at least in part on the change in contact.

14. The system of claim 13, wherein the user device is configured to detect the change in the PPG signal by being configured to:
determine that a direct current (DC) level of the PPG signal has changed by a threshold amount within a threshold duration of time.

15. The system of claim 13, wherein the reference cardiac pulse waveforms are based at least in part on a portion of the PPG signal measured by the wearable device before the change in contact.

16. The system of claim 13, wherein the change in the metric comprises a change in amplitude between the cardiac pulse waveforms, a change in skin or body temperature of the user, a change in acceleration of the wearable device, or a change in light reflected into a laser component.

17. The system of claim 13, wherein the user device is configured to determine the physiological metric by being configured to:
exclude a second portion of the PPG signal from a calculation to determine the physiological metric based at least in part on the second portion of the PPG signal coinciding with the change in contact.

18. The system of claim 13, wherein the user device is configured to determine the physiological metric by being configured to:
apply a weighting factor to a second portion of the PPG signal based at least in part on the second portion of the PPG signal coinciding with the change in contact.

19. The system of claim 13, wherein the user device is further configured to:
cause, based at least in part on the change in contact, a graphical user interface to display a message prompting the user to adjust an orientation of the wearable device.

20. The system of claim 19, wherein the PPG signal is associated with a first set of optical components, and wherein the user device is further configured to:
determine a difference in quality between the PPG signal and a second PPG signal associated with a second set of optical components, wherein the message to adjust the orientation is based at least in part on the difference in quality.

21. A method, comprising:
detecting a change in a photoplethysmogram (PPG) signal measured by a wearable ring device or a change in morphology between cardiac pulse waveforms that are based at least in part on the PPG signal and reference cardiac pulse waveforms;
determining, based at least in part on the change in the PPG signal or the change in morphology coinciding with a change in a metric measured by the wearable device, that the change in the PPG signal or the change in morphology is based at least in part on a change in contact between the wearable device and the skin of a user of the wearable device; and
determining, using at least a portion of the PPG signal, a physiological metric for the user based at least in part on the change in the PPG signal or the change in morphology being based at least in part on the change in contact.
